(19) United States
(12) Patent Application Publication
(10) Pub. No.: US 2015/0071857 A1
(43) Pub. Date: Mar. 12, 2015

(54) STEROID HORMONE AND CHOLESTEROL PATHWAYS AS ONE UNIFIED HOMEOSTATIC SYSTEM

(71) Applicant: LIGAND PHARMACEUTICALS, INC., La Jolla, CA (US)

(72) Inventor: Lin Zhi, San Diego, CA (US)

(21) Appl. No.: 14/382,943

(22) PCT Filed: Mar. 5, 2013
(86) PCT No.: PCT/US2013/029195

§ 371 Date: Sep. 4, 2014

Related U.S. Application Data
(60) Provisional application No. 61/607,991, filed on Mar. 7, 2012.

(51) Int. Cl. A61K 31/56; G01N 33/50

(52) U.S. Cl. CPC A61K 31/56; G01N 33/5041; G01N 2500/00
USPC 424/9.2; 514/170; 552/509; 435/29; 435/25; 435/15; 435/7.1; 435/11

(57) ABSTRACT

A unified homeostatic system of cholesterol and steroid hormone pathways is described, in which the uses or modulations of function of the homeostatic system of cholesterol and steroid hormone pathways are linked by lipoproteins, and are used or modulated to achieve a therapeutic benefit, to diagnose a disease or medical condition in humans, or to develop suitable active agents or combinations of active agents. Pharmaceutical compositions, methods of treatment, methods of drug development, and assay methods that rely on the new understanding of the homeostatic system are described.

### BACKGROUND OF THE INVENTION

1. Field of the Invention

This application relates to the homeostatic system of cholesterol and steroid hormone pathways. In particular, it relates to the uses or modulations of function of the homeostatic system of cholesterol and steroid hormone pathways linked by lipoproteins, and to uses or modulations of function of the homeostatic system of cholesterol and steroid hormone pathways to achieve a therapeutic benefit, to diagnose a disease or medical condition in humans, or develop suitable active agents or combinations of active agents.

2. Description of the Related Art

Circulating cholesterol is derived either by biosynthesis mainly in the liver from acetyl coenzyme A (CoA) or by absorption at the enterocyte of intestine from dietary and biliary sources. Cholesterol is cleared mainly in the liver through bile excretion or metabolism as the raw material for biosynthesis of bile acids that are ligands of nuclear receptor farnesoid X receptor (FXR). Cellular cholesterol is managed by many different lipoprotein particles generally classified as chylomicron (CM), VLDL, IDL, LDL and HDL according to their densities (FIG. 1). CM is involved in loading triglycerides and cholesterol absorbed at enterocyte of the intestine, delivering the triglycerides to muscle and fat tissues, and delivering the remaining cholesterol to the liver. Chylomicron remnant (CMR) is a CM form that has off-loaded most of its triglycerides. VLDL loads hepatic triglycerides and cholesterol, delivers the triglycerides to muscle and fat tissues, and is converted to IDL then LDL when its density is increased by off-loading triglycerides and up-loading cholesterol ester through CETP (Kwiterovich P O Jr. 2008 Recognition and management of dyslipidemia in children and adolescents. J. Clin. Endocrinol. Metab. 93:4200-4209). Key proteins of HDL are synthesized in the liver and intestine, and HDL matures by picking up cholesterol mainly in periphery tissues via a group of transporters. The lipoprotein particles deliver hydrophobic cholesterol ester molecules from the blood to cells by “docking” at the corresponding receptors on the surface of certain cell membranes. Due to the unique tissue-selective expression of the receptors, LDL particles deliver cholesterol obtained from the liver via VLDL and from HDL via CETP to periphery tissues, although majority of cholesterol in LDL goes back to the liver along with internalization of LDL that is controlled by hepatic cholesterol concentration (Goldstein J L, et al. 2001 The cholesterol quartet. Science 292:1310-1312), and HDL particles carry cholesterol selectively in the direction from peripheral to the liver in a process termed reverse cholesterol transport (RCT) (Khera A V, et al. 2010 Future therapeutic directions in reverse cholesterol transport. Curr. Atheroscler. Rep. 12:73-81).

Blood LDL cholesterol level (LDL-C) is positively correlated with coronary heart disease (CHD). Lowering LDL-C, whether by adapting to a healthy life-style or taking drugs that block cholesterol biosynthesis (or both), may significantly reduce atherosclerosis and the risk of developing CHD. Based on epidemiological studies showing an inverse correlation with CHD, blood HDL cholesterol level (HDL-C) has been considered as an independent risk factor of CHD. Research and development efforts exist to develop regimes to raise HDL-C to further lower CHD risk beyond LDL-C reduction management (Natarajan P, et al. 2010 High-density lipoprotein and coronary heart disease. JACC 55:1283-1299). Raising HDL-C via drug intervention, however, is still controversial and has yet to be proved clinically beneficial due to often unexpected clinical results and a lack of understanding of global HDL regulation.

Cholesterol is a raw material of steroid biosynthesis and a substantial portion of the supply of cholesterol for biosynthesis comes from circulating lipoproteins. The literature does not clearly establish whether LDL or HDL is mainly responsible for cholesterol delivery into steroidogenic organs such as adrenals, ovaries, and testes in humans. Steroidogenesis in adrenal glands have been investigated for this purpose, and it is generally accepted that in humans LDL handles supply of cholesterol for steroid biosynthesis and in rodents HDL is the main supplier (Miller M L, Auchus R J. 2011 The molecular biology, biochemistry, and physiology of human steroidogenesis and its disorders. Endocr. Rev. 32:81-151). LDL delivers cholesterol through LDL receptor (LDLR) mediated endocytosis along with internalization of LDL. HDL delivers cholesterol through the HDL receptor, scavenger receptor class B type I (SR-BI), without internalization of HDL. It has been demonstrated that adrenal steroid hormone production is normal in mice lacking LDLR (Kraemer F B, et al. 2007 The LDL receptor is not necessary for acute steroidogenesis in mouse adrenocortical cells. Am. J. Physiol. Endocrinol. Metab. 292:E408-E412), and that glucocorticoid synthesis in SR-BI-null mice is severely hampered in response to lipopolysaccharide, bacterial infection, stress, or ACTH (Cai L, et al. 2008 SR-BI protects against endotoxemia in mice through its roles in glucocorticoid production and hepatic clearance. J. Clin. Invest. 118:364-375). In human fetal adrenal tissues, LDL-C is involved in steroid synthesis (Brown M S, et al. 1979 Receptor-mediated uptake of lipoprotein-cholesterol and its utilization for steroid synthesis in the adrenal cortex. Recent Prog. Hormone Res. 35:215-257). In patients with LDLR mutations, however, adrenal function and steroid synthesis were normal (Illingworth D R, et al. 1984 Adrenocortical response to adrenocorticotropin in heterozygous familial hypercholesterolemia. J. Clin. Endocrinol. Metab. 58:206-211). In patients with complete LDLR deficiency, adrenal cortical function is only mildly affected (Illingworth D R, et al. 1983 Adrenal cortical function in homozygous familial hypercholesterolemia. Metabolism 32:1045-1052). After identification of HDL receptor in the mid-1990s, both LDL and HDL receptor genes are expressed in parallel in human adrenal tissues and both could be the source of cholesterol for steroid synthesis, although the receptor gene upregulation by ACTH was faster for LDL, and HDL only enhanced ACTH-induced cortisol production but not basal (Liu J, et al. 2000 Expression of low and high density lipoprotein receptor genes in human adrenals. Eur. J. Endocrinol. Jun. 1, 2000 142:677-682). A functional mutation in SR-BI has been identified in humans, and the reduced function of SR-BI was found to be associated with decreased adrenal steroidogenesis, which suggested that HDL fulfills an unanticipated role in human adrenal steroid synthesis (Vergeer M, et al. 2011 Genetic variant of the scavenger receptor BI in humans. N. Engl. J. Med. 364:136-145). The view that LDL-C is the major source of steroidogenesis in human adrenal gland is now being challenged (Connelly M A. 2009 SR-BI-mediated HDL cholesteryl ester delivery in the adrenal gland. Mol. Cell. Endocrinol. 300:83-88). Human SR-BI gene is expressed in testis and ovaries, and deficiency of SR-BI was found to cause sex hormone deficiency in human steroidogenic cells (Kolmakova A, et al. 2010 Deficiency of scavenger receptor class B type I negatively affects progesterone secretion in human granulosa cells. Endocrinol. 151:5519-5527). In the humans with a SR-BI mutation (Vergeer M, et al. 2011, see above), urinary steroid excretion is reduced, including total 17-ketogenic steroids (intermediates for synthesis of androgens and estrogens), 11-hydroxyandrosterone (a metabolite of DHT), and pregnanediol (a metabolite of progesterone) secretion. These results suggest that steroidogenesis in ovaries and testis may be suppressed when HDL cholesterol delivery is hindered by the SR-BI mutation.

Steroid acute regulatory protein (StAR) is responsible for transport of cholesterol in the cells to mitochondria for steroid hormone biosynthesis (FIG. 2). Mutations in the StAR gene cause cholesterol accumulation in the cytoplasm of the steroidogenic cells as large lipid droplets (Lin D, et al. 1995 Role of steroidogenic acute regulatory protein in adrenal and gonadal steroidogenesis. Science 267:1828-1831), which suggests that the cholesterol delivery may not be controlled by cholesterol concentration in the cells. Cholesterol is converted into the five distinct classes of steroid hormones by multiple enzymes and cofactors, in multiple pathways, in a tissue-selective and cell-selective fashion and the first step cleaving cholesterol side chain to form pregnenolone is the rate-limiting step (Miller M L, Auchus R J. 2011, see above). Aldosterone is produced in zona glomerulosa cells of adrenal gland via progesterone as the key intermediate, and regulated by feedback control of angiotensins and electrolytes. Cortisol is produced in zona fasciculate cells of adrenal gland via 17-hydroxyprogesterone (17OHP) as the main route and regulated by CRH/ACTH negative feedback through hypothalamic-pituitary-adrenal (HPA) axis. In the adrenal zona reticularis cells, dehydroepiandrosterone (DHEA) is the major product and is partially converted to testosterone. In testicular Leydig cells, testosterone (T) is synthesized via DHEA as the major pathways and controlled by negative feedback of peptide hormones, GnRH and LH, through hypothalamic-pituitary-gonad (HPG) axis. Small amount of estradiol (E2) is also produced via aromatization of T in Leydig cells. In ovaries, steroidogenesis is more complicated due to variation of the menstrual cycle and differences in enzyme distribution in cell types. Progesterone is synthesized in corpus luteum under the influence of LH as part of the negative HPG feedback loop and E2 is produced via DHEA and estrone as the major route under control of FSH in theca and granulose cells. Furthermore, estrogens in high concentration during follicular phase of the menstrual cycle have a positive feedback through HPG axis on top of the regular negative feedback mechanism (Hu L, et al. 2008 Converse regulatory functions of estrogen receptor-α and -β subtypes expressed in hypothalamic gonadotropin-releasing hormone neurons. Mol. Endocrinol. 22:2250-2259). Steroid hormone binding proteins, SHBG and CBG, play a critical role in steroid transportation in circulation and the protein levels determine the biologically active concentration of the hormones and thus can attenuate hormone feedback intensity quite differently depending on the specific biological circumstance. Moreover, SHBG may play an active role in human physiology more than a binding protein (Caldwell J D, Jirikowski G F. 2009 Sex hormone binding globulin and aging. Horm. Metab. Res. 41:173-182).

Steroid hormones thus play roles in reproduction, development, metabolism, immune response, fluid homeostasis, and aging, and the steroid hormone receptors have been targeted for medicine in many therapeutic areas. Development of selective steroid hormone receptor modulators would offer new generations of medicine to better provide benefits and avoid side effects of the natural steroid hormones. In contrast to the direct involvement of many non-steroid nuclear receptors (for example, thyroid and certain orphan receptors) in regulating lipid metabolism, storage, transport, and elimination (Chawla A, et al. 2001 Nuclear receptors and lipid physiology: opening the X-files. Science 294:1866-1870), steroid hormones have not fully-understood, complex relationships with lipids and the mechanisms of interaction are much less well-understood, thus limiting improvements of lipid profile for steroid hormone receptor modulators.

### SUMMARY OF THE INVENTION

This application describes a novel mechanism that lipoproteins, mainly HDL, dynamically link cholesterol homeostasis pathways and steroid hormone homeostasis pathways to function as a single homeostatic system. The unified homeostatic system of cholesterol and steroid hormone pathways linked by mainly HDL provides a unique, novel perspective in viewing the relationship of HDL-C and steroid hormones, and offers new insights to answer many outstanding questions in the fields of the cholesterol and steroid hormone homeostases. The steroid hormone and cholesterol pathways as one unified (SHAC1) homeostatic system can be used to develop new clinical methods of improving patient lipid profile and reducing cardiovascular risk, and to develop new generations of medicines with fewer side effects in treatment of disorders or conditions related to lipids, steroid hormones, and potentially metabolic pathways.

Some embodiments of the present invention include methods of diagnosing a disorder or a condition associated with the balance of the unified homeostatic system of cholesterol and steroid hormone pathways linked by lipoproteins in a patient. Some methods include testing a blood sample by existing and/or new blood chemistry testing protocols and determining the imbalance of the system and CHD risk based on data analysis, developed by considering the cholesterol and steroid hormone pathways as one unified homeostatic system. Some methods include a genetic testing to determine the imbalance of the system and CHD risk based on data analysis, developed by considering the SHAC1 homeostatic system as a whole.

Some embodiments of the present invention include methods of treating a disorder or a condition associated with the balance of the SHAC1 homeostatic system in a patient in need of such treatment. Some methods include administering an initial effective amount of a regiment that is developed based on control of the SHAC1 homeostatic system linked by lipoproteins, mainly HDL.

Some embodiments of the present invention include methods of managing the balance of the SHAC1 homeostatic system in a normal person in need of such treatment. Some methods include administering an initial effective amount of a regiment that is developed based on control of the SHAC1 homeostatic system.

In some embodiments, the disorder or condition is associated with dyslipidemia, dyscholesterolemia, dyslipoproteinemia, and/or atherosclerosis.

In some embodiments, the disorder or condition is associated with steroid hormone imbalance or steroid hormone management.

In some embodiments, the disorder or condition is associated with metabolic pathways.

In some embodiments, the disorder or condition is associated with pathophysiological state.

In some embodiments, the disorder or condition is associated with aging.

In some embodiments, the disorder or condition is associated with a medical intervention.

Other embodiments include methods that include treating a disorder associated with dyslipidemia, dyscholesterolemia, dyslipoproteinemia, and/or atherosclerosis with a regiment in a patient in need of such treatment. Some such methods include administering an effective amount of a non-peptidyl small molecule, a peptide, a biologic molecule, an antibody, an antisense molecule, a small interfering RNA molecule, a gene therapy, or stem cell therapy that improves HDL productivity in RCT by increasing cholesterol consumption for sterol biosynthesis.

Other embodiments include methods that include treating a disorder associated with dyslipidemia, dyscholesterolemia, dyslipoproteinemia, and/or atherosclerosis with the regiment in a patient described above in combination of LDL-C lowering agents, such as a statin drug, a bile acid sequestrant, and a cholesterol absorption inhibitor.

Other embodiments include methods that include treating a disorder or condition associated with steroid hormone imbalance with a regiment in a patient in need of such treatment. Some such methods include administering an effective amount of new generation of steroid hormone receptor modulators with improved lipid profile developed by considering the SHAC1 homeostatic system.

Some embodiments include methods of intervening steroid hormone balance to achieve a medical purpose in humans. Some such methods include administering an effective amount of a hormonal regiment of a non-peptidyl small molecule, a peptide, a biologic molecule, an antibody, an antisense molecule, a small interfering RNA molecule, a gene therapy, or stem cell therapy that increase or does not decrease cholesterol consumption for sterol biosynthesis, and/or that does not increase the venous thrombosis risk.

Some embodiments include a pharmaceutical composition. In some embodiments this composition comprises a pharmaceutically active amount of a first compound. This first compound may be a steroid, a progenitor of a steroid, a regulator of a steroid, a modulator of a steroid receptor, and a pharmaceutically acceptable prodrug or salts thereof. The first compound may have a positive effect on a physiological process related to a steroid biosynthesis, turnover, localization, sensing or action, and the first compound may have a negative effect on cholesterol homeostasis, lipoprotein homeostasis, or cholesterol-related lipid homeostasis,

In some embodiments, the composition may further comprise a pharmaceutically active amount of a second compound. This second compound may be a steroid, a progenitor of a steroid, a regulator of a steroid, a modulator of a steroid receptor, and a pharmaceutically acceptable prodrug or salt thereof, a cholesterol biosynthesis modulator, a cholesterol accumulation modulator, a cholesterol transport modulator, a cholesterol-related lipid biosynthesis modulator, a cholesterol related lipid accumulation modulator, a lipoprotein modulator, and pharmaceutically acceptable prodrug or salt thereof.

The second compound may not substantially interfere with the positive effect of the first compound, and the second compound may exhibit an effect antagonistic to the negative effect of the first compound.

The pharmaceutical composition may further comprise at least one pharmaceutically acceptable carrier or diluent.

In some embodiments, the first compound may be selected from the group comprising (a) adrenocorticotropin, (b) aldosterone, (c) an androgenic-anabolic steroid, (d) an androgen, (e) an AR antagonist, (f) a cytochrome b5 (CYB5A) or an activity regulator thereof, (g) DHEA, (h) DHEA sulfate, (i) ethinyl estradiol, (j) estradiol, (k) natural or synthetic estrogen, (l) esterified estrogen, (m) a GnRH modulator, (n) 11-hydroxyandrosterone, 17-hydroxyprogesterone, (o) a 17-ketogenic steroid, (p) levonorgestrel, (q) medroxyprogesterone acetate, (r) a P450-oxidoreductase (POR) or an activity regulator thereof, (s) a P450c17 (CYP17A1) phosphorylation regulator, (t) pregnanediol, (u) pregnenolone, (v) progestin, (w) a steroid hormone receptor modulator, (x) a steroidal androgen, (y) a SERM compound, and (z) an SHBG or SHBG regulator.

In some embodiments, the composition has a first compound that exhibits a negative effect on HDL levels, and a second compound that increases HDL levels without substantially interfering with the positive effect of the first compound.

In some embodiments, first compound is an oral androgenic-anabolic steroid, progestin, high-dose isoflavone, cortisol, gonadotropin inhibitor, androgen synthesis inhibitor, aldosterone, SR-BI inhibitor, 21α-hydroxylase inhibitor, 11β-hydroxylase inhibitor, or a steroid binding globulin inhibitor, and the second compound is an omega-3 acid ethyl ester, statin, oral estrogen, dexamethasone, CETP inhibitor, total testosterone, non-orally administered androgen, corticosteroid, MR agonist inhibitor, GnRH modulator, steroid binding globulin (SHBG and CBG), or endogenous steroid biosynthesis promoter.

In some embodiments, the composition's first compound is a steroid, steroid biosynthesis regulator, steroid stability regulator, steroid localization regulator or steroid signaling-regulating molecule, and the second compound is a selective steroid receptor modulator (SSRM).

In some embodiments, the first compound is a tissue-specific SARM such as LGD-3303 and the second compound is a SERM compound.

In some embodiments, the second compound exhibits at least one of the following: heightened liver antagonistic activity, heightened hypothalamic antagonistic activity, heightened pituitary gland antagonistic activity, specific liver antagonistic activity, specific hypothalamic antagonistic activity, and specific pituitary gland antagonistic activity.

In some embodiments, the first compound is a progesterone and the second compound is an SPRM that reduces a stimulative effect of progesterone on breast tissues without impacting an anti-estrogenic effect of said progesterone in the uterus.

In some embodiments, the first compound is an estrogen and said second compound is an SSRM that reduces a venous thrombosis negative effect of said first compound.

In some embodiments, the second compound is a statin.

Some embodiments include a pharmaceutical composition. In some embodiments the composition includes a pharmaceutically active amount of a first compound. This first compound may a cholesterol regulator, a cholesterol-related lipid regulator, a lipoprotein regulator, or pharmaceutically acceptable prodrugs or salts thereof. The first compound may have a positive effect on a physiological process related to (a) cholesterol biosynthesis, (b) cholesterol turnover, (c) cholesterol localization, (d) cholesterol sensing, (e) cholesterol action, (f) cholesterol-related lipid biosynthesis, (g) cholesterol-related lipid turnover, (h) cholesterol-related lipid localization, (i) cholesterol-related lipid sensing, (j) lipoprotein homeostasis, (k) cholesterol metabolism, or (l) lipoprotein action, and the first compound may have a negative effect on steroid homeostasis.

In some embodiments, the composition may further comprise a second compound which may be (a) a steroid, (b) a progenitor of said steroid, (c) a regulator of said steroid, (d) a regulator of the synthesis or accumulation of said steroid, (e) a regulator of signal transduction related to said steroid, and (f) a modulator of said steroid receptor, and pharmaceutically acceptable prodrugs or salts thereof. The second compound may exhibit an effect antagonistic to the negative effect of the first compound.

In some embodiments, the composition may further comprise at least one pharmaceutically acceptable carrier or diluent.

In some embodiments, the first compound is (a) a bile acid sequestrant, (b) a cholesterol absorption inhibitor, (c) a cortisol, (d) a CETP inhibitor, (e) dexamethasone, (f) an estrogen or progestrin that impacts HLD levels, (g) a GnRH modulator, (h) an isoflavone, (i) a long-term calorie restriction regime, (j) medroxyprogesterone acetate, (k) an omega-3 acid ethyl ester, or (l) a statin.

In some embodiments, the first compound is a statin.

In some embodiments, the second compound is an SSRM.

In some embodiments, the first compound is a liver-targeting SHBG modulator and the second compound enhances SHBG binding to steroids but does not interfere with the positive effect of the first compound. The positive effect can include increasing HDL levels and increasing HDL efficiency, and the second compound may exhibit an effect antagonistic to the negative effect of the first compound of decreasing said endogenous steroid hormone production. The second compound to this liver targeting SHBG modulator is an SSRM.

Some embodiments include a method for altering (a) steroid accumulation level, (b) steroid localization, (c) steroid sensing, or (d) steroid signal transduction, in at least one cell, tissue, organ or region of a mammal. In some aspects the method comprises identifying a mammal having a condition associated with the trait and administering to the mammal a first compound or regimen that alters said accumulation level. In some aspects the embodiments further comprise administering a second compound or regimen. In some aspects the second compound does not interfere with a desired effect on said first trait, and the second compound exhibits an effect on the biosynthesis, accumulation or transport of cholesterol, HDL or LDL that is antagonistic to the effect of the first compound.

In some embodiments, the first compound is (a) adrenocorticotropin, (b) aldosterone, (c) an androgenic-anabolic steroid, (d) an androgen, (e) an AR antagonist, (f) a cytochrome b5 (CYB5A) or an activity regulator thereof, (g) DHEA, (h) DHEA sulfate, (i) ethinyl estradiol, (j) estradiol, (k) natural or synthetic estrogen, (l) esterified estrogen, (m) a GnRH modulator, (n) 11-hydroxyandrosterone, 17-hydroxyprogesterone, (o) a 17-ketogenic steroid, (p) levonorgestrel, (q) medroxyprogesterone acetate, (r) a P450-oxidoreductase (POR) or an activity regulator thereof, (s) a P450c17 (CYP17A1) phosphorylation regulator, (t) pregnanediol, (u) pregnenolone, (v) progestin, (w) a steroid hormone receptor modulator, (x) a steroidal androgen, (y) a SERM compound, or (z) an SHBG or SHBG regulator.

In some embodiments, the second compound is a statin. In some, the second compound is an SSRM. In some, the second compound has heightened activity in the liver, hypothalamus, or pituitary gland of the mammal.

In some embodiments, the administering occurs at substantially the same time.

Some embodiments include a method for altering a trait such as (a) cholesterol accumulation level, (b) cholesterol localization, (c) cholesterol sensing, (d) cholesterol signal transduction, or (e) lipoprotein accumulation level, in at least one cell, tissue, organ or region of a mammal. In some aspects this comprises identifying a mammal having a condition associated with the trait, administering to the mammal a first compound or regimen that alters the first trait, and administering to the mammal a second compound or regimen. In some aspects the second compound or regimen does not interfere with a desired effect on the regulation of the trait, and the second compound exhibits an effect on a second trait such as (a) steroid biosynthesis, (b) steroid accumulation, (c) steroid transport, (d) steroid signaling or (e) steroid sensing, that is antagonistic to the effect of said first compound.

Some embodiments include a method for altering a trait such as (a) cholesterol-related lipid accumulation level, (b) cholesterol-related lipid localization, (c) cholesterol-related lipid sensing, (d) cholesterol-related lipid transport, or (e) lipoprotein accumulation level, in at least one cell, tissue, organ or region of a mammal. In some aspects the method comprises identifying a mammal having a condition associated with the first trait. In some aspects the method comprises administering to the mammal a first compound or regimen that alters the cholesterol-related lipid accumulation level and administering to the mammal a second compound or regimen. In some aspects the second compound or regimen does not interfere with an effect on the regulation of the accumulation level. In some aspects the second compound exhibits an effect on steroid biosynthesis, accumulation or transport that is antagonistic to the effect of the first compound.

In some embodiments, the method comprises a regime having a first compound or regime consisting of (a) a bile acid sequestrant, (b) a cholesterol absorption inhibitor, (c) a cortisol, (d) a CETP inhibitor, (e) dexamethasone, (f) an estrogen or progestrin that impacts lipid levels, (g) a GnRH modulator, (h) an isoflavone, (i) a long-term calorie restriction regime, (j) medroxyprogesterone acetate, (k) an omega-3 acid ethyl ester, or (l) a statin.

In some embodiments, the regulator is a statin. In some aspects the second compound is an SSRM.

Some embodiments include a method for altering at least one cholesterol accumulation level in at least one cell, tissue, organ or region of a mammal. In some aspects of this embodiment the method comprises identifying a mammal having a condition associated with the cholesterol accumulation level and administering to the mammal a first compound or regimen that alters the cholesterol accumulation level. In some aspects the first compound is a steroid synthesis regulator.

In some embodiments, the compound is a) DHEA, (b) DHEAS, (c) artificial adrenarch, (d) a 17,20-lyase activity regulator, (e) a P450-oxidoreductase (POR), (f) a P450-oxidoreductase (POR) regulator, (g) a cytochrome b5 (CYB5A), (h) a cytochrome b5 (CYB5A) regulator, (i) a P450c17 (CYP17A1) protein kinase, or (j) a P450c17 (CYP17A1) protein kinase regulator.

Some embodiments include a method of identifying at least one agent of a multi-agent medicament for altering steroid accumulation level, steroid sensing, steroid localization, steroid action, and/or steroid signal transduction, in at least one cell, tissue, organ or region of a mammal. In some aspects the method comprises administering a first agent to the mammal, wherein the first agent affects at least one member of the list above and administering a second agent to the mammal, evaluating whether the second agent counteracts the effect on at least one member of said group, and evaluating whether the second agent exhibits an effect on cholesterol biosynthesis, cholesterol accumulation, cholesterol transport, cholesterol-related lipid synthesis, cholesterol-related lipid accumulation, lipoprotein homeostasis, or cholesterol-related lipid transport, that is antagonistic to the effect of the first agent.

In some embodiments, the second compound is (a) a non-peptidyl small molecule, (b) a peptide, (c) an antibody, (d) an antisense molecule, (e) a small interfering RNA molecule, (f) a gene, or (g) a stem cell.

In some embodiments, the first compound is (a) adrenocorticotropin, (b) aldosterone, (c) an androgenic-anabolic steroid, (d) an androgen, (e) an AR antagonist, (f) a cytochrome b5 (CYB5A) or an activity regulator thereof, (g) DHEA, (h) DHEA sulfate, (i) ethinyl estradiol, (j) estradiol, (k) natural or synthetic estrogen, (l) esterified estrogen, (m) a GnRH modulator, (n) 11-hydroxyandrosterone, 17-hydroxyprogesterone, (o) a 17-ketogenic steroid, (p) levonorgestrel, (q) medroxyprogesterone acetate, (r) a P450-oxidoreductase (POR) or an activity regulator thereof, (s) a P450c17 (CYP17A1) phosphorylation regulator, (t) pregnanediol, (u) pregnenolone, (v) progestin, (w) a steroid hormone receptor modulator, (x) a steroidal androgen, (y) a SERM compound, or (z) an SHBG or SHBG regulator.

In some embodiments, the first molecule affects androgen signaling and the second molecule is a SARM. In some aspects the first molecule affects estrogen signaling and the second molecule is an SERM. In some aspects the first molecule affects progesterone signaling and said second molecule is an SPRM.

In some embodiments, a molecule is identified by the process above.

Some embodiments include a method of identifying at least one agent of a multi-agent medicament for altering cholesterol accumulation level in at least one cell, tissue, organ or region of a mammal. In some embodiments, the method comprises administering a first agent to the mammal, wherein the first agent affects said cholesterol accumulation level, administering a second agent to the mammal, evaluating whether the second agent does not counteract the effect on the regulation of the cholesterol accumulation level of the first compound, and evaluating whether the second agent exhibits an effect on steroid biosynthesis, accumulation or transport which is antagonistic to the effect of the first agent.

Some embodiments include a method of identifying at least one agent of a multi-agent medicament for altering at least one cholesterol-related lipid accumulation level in at least on cell, tissue, organ or region of a mammal. In some embodiments, the method comprises administering a first agent to the mammal. In some aspects of the embodiments the first agent affects cholesterol-related lipid accumulation level, cholesterol-related lipid localization, cholesterol-related lipid sensing, lipoprotein homeostasis, and cholesterol-related lipid signaling. The method may comprise administering a second agent to the mammal, evaluating whether the second agent does not counteract the effect of the first agent, and evaluating whether the second agent exhibits an effect on steroid biosynthesis, steroid accumulation, steroid transport, steroid sensing, steroid action, and steroid signaling, wherein the effect of the second agent is antagonistic to the effect of the first agent.

In some embodiments, the regulator is (a) a bile acid sequestrant, (b) a cholesterol absorption inhibitor, (c) a cortisol, (d) a CETP inhibitor, (e) dexamethasone, (f) an estrogen or progestrin that impacts HLD levels, (g) a GnRH modulator, (h) an isoflavone, (i) a long-term calorie restriction regime, (j) medroxyprogesterone acetate, (k) an omega-3 acid ethyl ester, or (l) a statin.

In some embodiments, a compound is identified through the process above.

Some embodiments include a method of identifying a compound that both (a) modulates cholesterol synthesis, cholesterol turnover, cholesterol transport, cholesterol sensing, cholesterol metabolism, or cholesterol signaling, and (b) modulates steroid biosynthesis, steroid turnover, steroid localization, steroid transport, steroid sensing, or steroid signaling. In some aspects the method comprises monitoring the effect of the compound on a trait from the first group; monitoring the effect of the compound on a trait from the second group; and identifying the compound.

In some embodiments, a compound is identified through such a process.

Some embodiments include a method of identifying an effect of a steroid-modulating compound on cholesterol homeostasis that involves monitoring the effect of the compound on cholesterol homeostasis.

Some embodiments include a method of identifying an effect of a steroid-turnover regulating compound on cholesterol homeostasis that involves monitoring the effect of the compound on cholesterol homeostasis.

Some embodiments include a method of identifying an effect of a steroid-localization regulating compound on cholesterol homeostasis that involves monitoring the effect of the compound on cholesterol homeostasis.

Some embodiments include a method of identifying an effect of a steroid-sensing modulating compound on cholesterol homeostasis that involves monitoring the effect of the compound on cholesterol homeostasis.

In some embodiments, the steroid-modulating compound is (a) DHEA, (b) DHEAS, (c) artificial adrenarch, (d) a 17,20-lyase activity regulator, (e) a P450-oxidoreductase (POR), (f) a P450-oxidoreductase (POR) regulator, (g) a cytochrome b5 (CYB5A), (h) a cytochrome b5 (CYB5A) regulator, (i) a P450c17 (CYP17A1) protein kinase, (j) a P450c17 (CYP17A1) protein kinase regulator, or (k) a SSRM.

In some embodiments, a compound is identified through the above process.

Some embodiments include a method of identifying an effect of a compound on cholesterol homeostasis. In some aspects the method comprises monitoring the effect of the compound on cholesterol homeostasis, wherein the compound also modulates steroid synthesis, turnover, transport or sensing.

In some embodiments, the compound is (a) DHEA, (b) DHEAS, (c) artificial adrenarch, (d) a 17,20-lyase activity regulator, (e) a P450-oxidoreductase (POR), (f) a P450-oxidoreductase (POR) regulator, (g) a cytochrome b5 (CYB5A), (h) a cytochrome b5 (CYB5A) regulator, (i) a P450c17 (CYP17A1) protein kinase, and (j) a P450c17 (CYP17A1) protein kinase regulator, or (k) an SSRM.

In some embodiments, a compound is identified through the above process.

Some embodiments include a method of identifying an effect of a compound on SHBG levels. In some aspects the method comprises monitoring the effect of the compound on SHBG levels, wherein the compound is a nuclear receptor ligand, SSRM, PPAR modulator, or other SHBG regulator.

In some embodiments, a compound is identified through the above process.

Some embodiments include a method of identifying an effect of a molecule on endogenous sex steroid production comprising screening the molecule in an SHBG binding assay for an enhanced binding of an SHBG to a hormone, wherein the increased binding leads to a higher availability of cholesterol in steroidogenic tissues for steroid biosynthesis.

In some embodiments, a compound is identified through the above process.

Some embodiments include a method of identifying an effect of a steroid-modulating compound on lipoprotein homeostasis. In some aspects the method comprises monitoring the effect of the compound on lipoprotein homeostasis.

Some embodiments include a method of identifying an effect of a steroid-turnover regulating compound on lipoprotein homeostasis that involves monitoring the effect of the compound on lipoprotein homeostasis.

Some embodiments include a method of identifying an effect of a steroid-localization regulating compound on lipoprotein homeostasis that involves monitoring the effect of the compound on lipoprotein homeostasis.

Some embodiments include a method of identifying an effect of a steroid-sensing modulating compound on lipoprotein homeostasis that involves monitoring the effect of the compound on lipoprotein homeostasis.

In some embodiments, the steroid-modulating compound is (a) DHEA, (b) DHEAS, (c) artificial adrenarch, (d) a 17,20-lyase activity regulator, (e) a P450-oxidoreductase (POR), (f) a P450-oxidoreductase (POR) regulator, (g) a cytochrome b5 (CYB5A), (h) a cytochrome b5 (CYB5A) regulator, (i) a P450c17 (CYP17A1) protein kinase, (j) a P450c17 (CYP17A1) protein kinase regulator, or (k) a SSRM.

In some embodiments, a compound is identified through the above process.

Some embodiments include a method of identifying an effect of a compound on lipoprotein homeostasis. In some aspects the method comprises monitoring the effect of the compound on lipoprotein homeostasis, wherein the compound also modulates steroid synthesis, turnover, transport or sensing.

In some embodiments, the compound is (a) DHEA, (b) DHEAS, (c) artificial adrenarch, (d) a 17,20-lyase activity regulator, (e) a P450-oxidoreductase (POR), (f) a P450-oxidoreductase (POR) regulator, (g) a cytochrome b5 (CYB5A), (h) a cytochrome b5 (CYB5A) regulator, (i) a P450c17 (CYP17A1) protein kinase, and (j) a P450c17 (CYP17A1) protein kinase regulator, or (k) an SSRM.

In some aspects a compound is identified through the above process.

### BRIEF DESCRIPTION OF THE DRAWINGS

FIG. 1 shows schematically cholesterol homeostatic pathways in an abbreviated version including the known cholesterol sources and disposal routes.

FIG. 2 shows schematically steroid hormone homeostatic pathways in an abbreviated version including major known feedback loops and major steroid hormone biosynthesis steps omitting the enzymes and many intermediates.

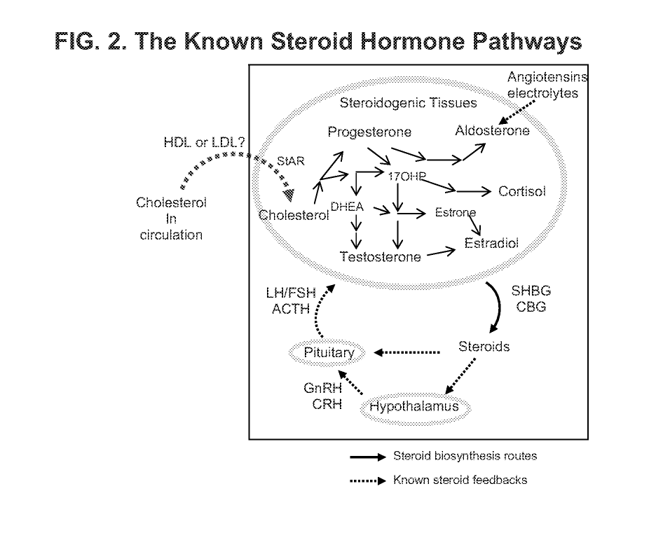

FIG. 3 shows the novel mechanism that lipoproteins, mainly HDL, link the cholesterol and steroid hormone pathways to function as one unified homeostatic system.

FIG. 4 shows the novel interchangeable relationship between HDL quantity and HDL capacity to transport cholesterol under strict endocrine control.

FIG. 5 shows the novel interchangeable relationship between LDL quantity and LDL capacity to transport cholesterol controlled by hepatic cholesterol output.

FIG. 6 shows the novel complementary steroid hormone feedback mechanism through the liver.

FIG. 7 shows the novel underlying relationships between CHD risk and the major risk factors based on equations (1) and (3).

FIG. 8 shows the known and novel lipid management strategies.

### DETAILED DESCRIPTION OF PREFERRED EMBODIMENTS

### Definitions

In accordance with the present invention and as used herein, the following terms are defined with the following meanings, unless explicitly stated otherwise.

The term “patient” refers to an animal being treated including a mammal, such as a dog, a cat, a cow, a horse, a sheep, and a human. Another aspect includes a mammal, both male and female.

The terms “treating” or “treatment” of a disease includes inhibiting the disease (slowing or arresting its development), providing relief from the symptoms or side-effects of the disease (including palliative treatment), and relieving the disease (causing regression of the disease). Non-limiting examples of treatments include medicines, changes to diet, and changes to exercise.

The term “positive effect” refers to desirable or beneficial effect to a patient.

The term “negative effect” refers to undesirable or harmful effect to a patient.

The term “a progenitor of steroid” refers to a molecule which is structurally similar to a steroid, which lacks the signaling potency of a given steroid and which may be converted into said steroid upon undergoing one or more chemical reactions.

The term “a regulator of steroid” refers to a molecule or signal that influences the signaling efficacy of a steroid. This influence may be accomplished by, for example, altering the stability, rate of synthesis, rate of degradation, localization, chemical structure or bioaccessability of a steroid, or by similarly affecting the stability, rate of synthesis, rate of degradation, activity, localization or other property of a receptor of a given steroid or a component in a pathway involved in the transduction of information conveyed by a steroid.

The term “a modulator of steroid receptor” refers to an agent selected from a small molecule, peptidyl, protein, antibody, antisense, stem-cell, a small interfering RNA molecule, a gene, a signal or others that modulates one or more steroid receptors including subtypes.

The term “cholesterol homeostasis” refers to the ability of an organism or system to maintain cholesterol at a certain level at a certain subcellular, cellular, tissue or organism-wide scale. It involves the regulation of cholesterol synthesis, degradation, and localization.

The term “lipoprotein homeostasis” refers to the ability of an organism or system to maintain lipoproteins at a certain level at a certain subcellular, cellular, tissue or organism-wide scale. It involves the regulation of lipoprotein synthesis, degradation, and localization.

The term “cholesterol-related lipid homeostasis” refers to the ability of an organism or system to maintain cholesterol-related lipids at a certain level at a certain subcellular, cellular, tissue or organism-wide scale. It involves the regulation of cholesterol-related lipid synthesis, degradation, and localization. A non-limiting list of examples of cholesterol-related lipids includes chylomicron (CM), VLDL, IDL, LDL and HDL.

The term “a cholesterol biosynthesis modulator” refers to a molecule or chemical signal that affects the rate of cholesterol biosynthesis by, for example, catalyzing cholesterol biosynthesis, affecting the rate of activity of enzymes that catalyze cholesterol biosynthesis, the accumulation levels of such enzymes, the availability or localization of said enzymes, the localization or accumulation levels of cholesterol precursors, the activity of enzymes that catalyze the degradation of precursors or of enzymatic catalytic molecules, or otherwise influences the rate of cholesterol biosynthesis.

The term “a cholesterol accumulation modulator” refers to a molecule or chemical signal that affects cholesterol accumulation levels by, for example, catalyzing cholesterol synthesis or degradation, affecting the rate of activity of enzymes that catalyze cholesterol synthesis or degradation, the accumulation levels of such enzymes, the availability or localization of said enzymes, the localization or accumulation levels of cholesterol precursors or degradation products, the activity of enzymes that catalyze the degradation of precursors or degradation products or of enzymatic catalytic molecules, or otherwise influences the rate of cholesterol biosynthesis and degradation or localization in such a way as to influence accumulation levels.

The term “a cholesterol transport modulator” refers to a molecule or chemical signal that affects cholesterol localization within a subcellular region, among cells, among tissues or at a whole organism level.

The term “a cholesterol-related lipid biosynthesis modulator” refers to a molecule or chemical signal that affects the rate of cholesterol-related lipid biosynthesis by, for example, catalyzing cholesterol-related lipid biosynthesis, affecting the rate of activity of enzymes that catalyze cholesterol-related lipid biosynthesis, the accumulation levels of such enzymes, the availability or localization of said enzymes, the localization or accumulation levels of cholesterol-related lipid precursors, the activity of enzymes that catalyze the degradation of precursors or of enzymatic catalytic molecules, or otherwise influences the rate of cholesterol-related lipid biosynthesis.

The term “a lipoprotein regulator” refers to a molecule or chemical signal that directly or indirectly affects the accumulation level, activity or localization of a lipoprotein.

The term “HDL efficiency” refers to overall capacity of a fixed HDL unit in transport of cholesterol.

The term “cholesterol accumulation level” refers to the net number of molecules or concentration of cholesterol in a given subcellular region, cell, extracellular space, tissue or whole organism. The accumulation level represents the aggregate effects of biosynthesis, localization, and degradation on a molecular population.

The term “lipoprotein accumulation level” refers to the net number of molecules or concentration of lipoproteins in a given subcellular region, cell, extracellular space, tissue or whole organism. The accumulation level represents the aggregate effects of biosynthesis, localization, and degradation on a molecular population.

The term “cholesterol-related lipid accumulation level” refers to the net number of molecules or concentration of cholesterol-related lipids in a given subcellular region, cell, extracellular space, tissue or whole organism. The accumulation level represents the aggregate effects of biosynthesis, localization, and degradation on a molecular population.

The term “a steroid synthesis regulator” refers to a molecule or chemical signal that influences the rate of synthesis of a steroid. Such a regulator may act by, for example, catalyzing chemical changes in a progenitor of a steroid, or by influencing the rate of activity of an enzyme that catalyzes such a change, or by influencing the accumulation level of any of the aforementioned enzymes or progenitors.

The term “steroid sensing” refers to the ability of a subcellular region, cell, tissue or whole-organism to assess the accumulation of a steroid. Such sensing may comprise both signal transduction triggered by the steroid and signal-transduction independent evaluation of steroid levels.

The term “steroid action” refers to biological activity of a steroid compound.

The term “a steroid-modulating compound” refers to a molecule or signal that affects steroid activity or accumulation levels by, for example, catalyzing steroid synthesis or degradation, affecting the rate of activity of enzymes that catalyze steroid synthesis or degradation, the accumulation levels of such enzymes, the availability or localization of said enzymes, the localization or accumulation levels of steroid precursors or degradation products, the activity of enzymes that catalyze the degradation of precursors or degradation products or of enzymatic catalytic molecules, or otherwise influences the rate of steroid biosynthesis and degradation in such a way as to influence accumulation levels, or for example, binding or modifying a steroid to affect its signaling capacity, altering a steroid localization to remove it from its receptor, or binding, modifying or affecting the accumulation level or localization of a steroid receptor or other component in a steroid signaling pathway to effect a change in the effect of a steroid on a cell, tissue or whole organism.

The present invention describes a novel mechanism that lipoproteins, mainly HDL, dynamically link cholesterol homeostasis pathways and steroid hormone homeostasis pathways to function as one unified homeostatic system. The present invention relates to methods of diagnosing a disorder or a condition associated with balance of the SHAC1 homeostatic system in a patient. The present invention also relates to methods of treating a disorder or condition associated with balance of the SHAC1 homeostatic system in a patient in need of such treatment. In some embodiments, certain compounds and compositions of a therapy that improves HDL productivity in RCT by modulating cholesterol consumption for steroid hormone biosynthesis include endogenous steroid hormone biosynthesis stimulating agents developed based on the two pathways of cholesterol and steroid hormone homeostases to function as one unified system.

### SHAC1 Homeostatic System

Current research in the cholesterol and steroid hormone fields are predominantly done independently and many basic questions remain unanswered. There is no report in the literature that addresses the outstanding issues of cholesterol and steroid hormones from a perspective of considering the two pathways as one system.

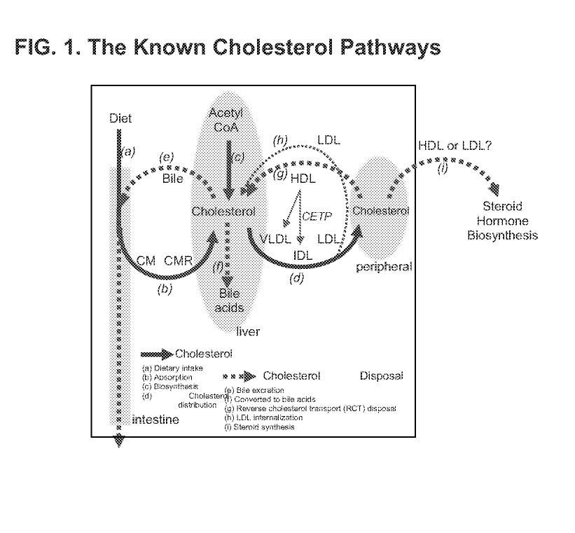

It has been known that hepatic cholesterol is positively correlated with LDL-C and, however, the fundamental cause of the correlation is not well defined. The current view emphasizes cholesterol's need of the liver as the cause, where, when hepatic cholesterol levels fall, LDLR gene transcription is induced, LDL-C is taken up more rapidly for internalization to release cholesterol in the liver, and the amount of LDL in plasma falls (Goldstein J L, et al. 2001, see above). Alternatively, lipoprotein particles can be viewed as different types of “vehicles” to move lipids around to meet the metabolic needs of cells and tissues. The correlation can be viewed from the need of delivering hepatic cholesterol to be available for peripheral tissues and the LDL internalization is the result but not the cause. In this view, the purpose of the lipoproteins to deliver hepatic cholesterol is very clear: the higher the cholesterol level in the liver, the more “vehicles” needed to move them around. As a result, when hepatic cholesterol levels fall, blood VLDL and IDL cholesterol, and triglyceride levels fall along with LDL-C. Nevertheless, LDL is clearly regulated by hepatic cholesterol output. The purpose of HDL has not been defined at all. Global regulation of HDL is not known and cannot be rationalized within the cholesterol homeostatic pathways (FIG. 1). The complex lesions of atherosclerosis are formed by LDL-C trafficking in a slow process, and elevation of LDL-C and buildup of the lesions do not seem to have any immediate disruption of a biological function or any direct control of HDL-C. Thus, the anti-atherogenic activity of HDL is rather a biological property than a purpose. At the cellular level, cholesterol can be stored in its ester form as cytoplasmic lipid droplets in peripheral cells as part of the intracellular cholesterol homeostasis. The cellular cholesterol pool can be accessed by HDL particles but is clearly not a determinant of HDL-C, and thus, there is no “excess” cholesterol in peripheral cells for HDL to dispose of as generally believed. In liver, FXR acting as a bile acid sensor controls bile acid homeostasis by regulating its target genes involved in bile acid disposal and biosynthesis from cholesterol, and has no direct control of HDL-C(Handschin C, Meyer U A. 2005 Regulatory network of lipid-sensing nuclear receptors: Roles for CAR, PXR, LXR, and FXR. Arch. Biochem. Biophys. 433:387-396). Oxysteroid biosynthesis is also a consumer of cholesterol but oxysteroids are synthesized in many different tissues and activate liver X receptor (LXR) as part of the machinery of hepatic lipid homeostasis, and the pathway does not seem to require or affect RCT. Steroid hormone biosynthesis in steroidogenic tissues is therefore the only remaining major known pathway of cholesterol metabolism that potentially regulates HDL.

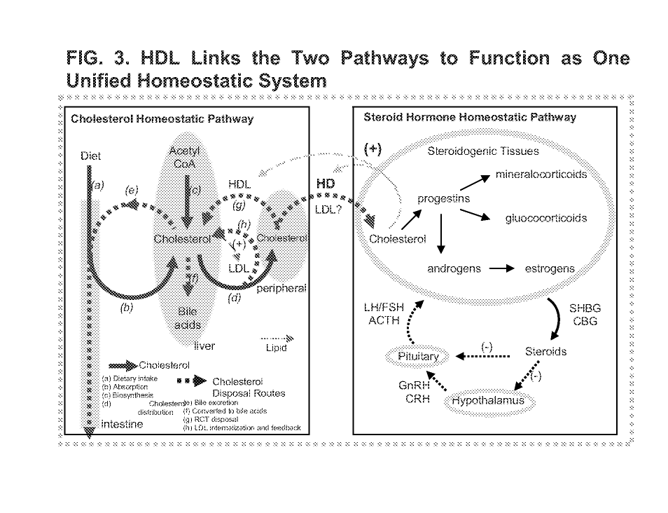

Based on review and analysis of the related epidemiological observations and clinical results in the literature, the present invention postulates that lipoproteins, mainly HDL, form a dynamic link between cholesterol and steroid hormone homeostatic pathways to function as one unified system (FIG. 3). Under the novel mechanism, cholesterol uptake amount from circulation for steroidogenesis regulates HDL, and influences LDL. Since steroidogenesis is under strict endocrine control, overall productivity of HDL in cholesterol transportation is controlled by steroid hormone homeostasis. LDL may play a redundant or complementary role in delivery of cholesterol to steroidogenic tissues and the feedback control of LDL via steroid hormone homeostasis seems to be minimal, if any, in comparison with via hepatic cholesterol output control. Any perturbation from either the cholesterol or the steroid hormone sides would result in a corresponding change of HDL productivity to balance the needs of the SHAC1 homeostatic system. When more endogenous steroids are needed, HDL level would be up-regulated to supply more cholesterol to the steroidogenic tissues, and when less steroids are needed, HDL level would be down-regulated. When cholesterol consumption for steroid synthesis affects the cholesterol amount in circulation, LDL level would be affected indirectly. When HDL-C or HDL productivity is up, more cholesterol would be consumed, and thus LDL-C or LDL productivity would be reduced. When HDL-C or HDL productivity is down, LDL-C or LDL productivity would be increased. By considering the SHAC1 homeostatic system, many outstanding questions related to cholesterol and steroid hormone pathways can be answered.

### Lipoprotein Quantity and Quality Exchange and Control

Lipoproteins contain different subfractions that are not equal in their capacity of cholesterol transportation. Fluctuation of lipoprotein is always associated with the composition change of the subfractions and is controlled by many physiological and pathophysiological factors (Deeb S, et al. 2003 Hepatic lipase and dyslipidemia: interactions among genetic variants, obesity, gender, and diet. J. Lipid Res. 44:1279-1286). Due to the huge success of lipid lowering management through reductions in cholesterol/fat intake, cholesterol biosynthesis, and cholesterol absorption at enterocytes, the capacity of LDL in cholesterol transportation has not been brought up as a major topic. On the other hand, experimental therapies to raise HDL-C face significant challenges to demonstrate atheroprotective effects on top of LDL-C lowering therapies. As a result, more attention has been diverted to the capacity of HDL as a determinant factor of atherosclerosis (Khera A V, et al. 2011 Cholesterol efflux capacity, high-density lipoprotein function, and atherosclerosis. N. Engl. J. Med. 364:127-135). However, the underlying relationship between lipoprotein quantity and capacity has not been described. Under the SHAC1 homeostatic system, lipoproteins are considered as the vehicles to transport and deliver cholesterol in circulation, and, as a result, they should be able to adjust the overall productivity in cholesterol transportation by adjusting number of the vehicles (HDL and LDL quantities) and the average deliverable cholesterol loads per vehicle (capacities of HDL and LDL) according to a specific physiological or pathophysiological state to meet the biological needs of cholesterol delivery. Based on the control of LDL-C by hepatic cholesterol output and control of HDL-C by steroid biosynthesis cholesterol needs, the relationships of HDL and LDL quantities and capacities to transport cholesterol can be expressed mathematically as

Ch_{Steroid}=f{HDL-C×C_{HDL}} (1)

Ch_{Liver}=f{LDL-C×C_{LDL}} (2)

where Ch_{steroid} is the cholesterol uptake amount from circulation for steroidogenesis, Ch_{Liver} is the cholesterol output amount of the liver, HDL-C and LDL-C are rough estimation of HDL and LDL quantities, C_{HDL} and C_{LDL} are the capacities of HDL and LDL in cholesterol transportation. By the equations, HDL or LDL can exchange their capacity for quantity or vise versa accordingly in each specific circumstance.

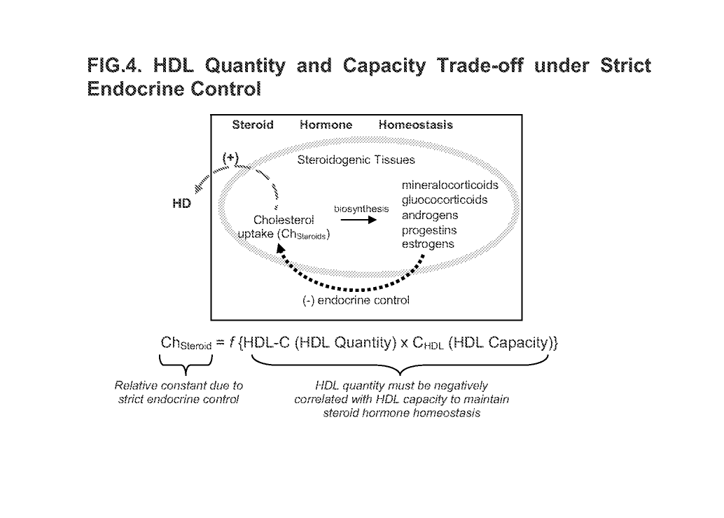

Since Steroidogenesis is under strict endocrine control. Thus, quantity and capacity of HDL, and their trade-off may be indirectly controlled by steroid hormone homeostasis (FIG. 4). When HDL quantity is changed by physiological, pathophysiological, or medical intervention, HDL capacity would change in opposite direction to maintain the steroid hormone homeostasis. When the capacity is hindered, HDL quantity would be increased to compensate for the reduced capacity. When steroid hormone biosynthesis is reduced in a pathophysiological state or by a medical treatment, normal HDL-C would indicate a reduction in HDL capacity.

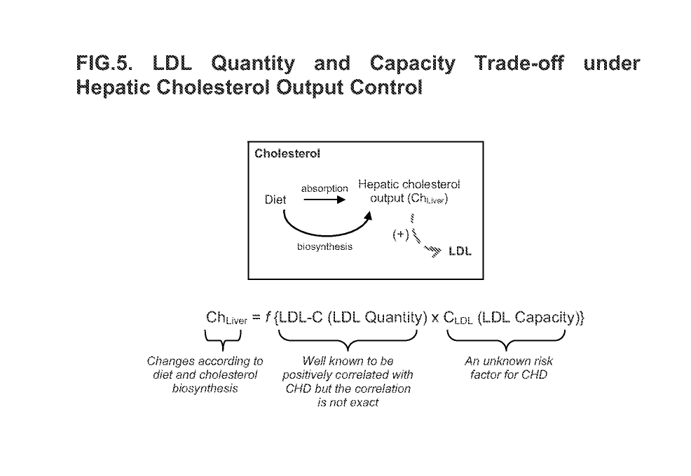

Hepatic cholesterol output amount, depending on diet, cholesterol biosynthesis, and cholesterol catabolism, can fluctuate in a wide range (FIG. 5). Quantity and capacity of LDL, and their trade-off are loosely controlled due to the bigger variation of hepatic cholesterol output. As a result, the range of LDL-C is much larger than that of HDL-C, and impact of C_{LDL} variation is probably unnoticeable in most cases. When the output is under certain control, LDL-C changes affected by a pathophysiological condition or medical treatment would be compensated by LDL capacity changes in an opposite direction.

### Effect of Perturbation of Cholesterol Homeostasis on HDL-C

The changes of HDL-C and steroid hormone levels in response to diet and statins can be explained by considering the SHAC1 homeostatic system. When LDL-C is increased by diet, more cholesterol would be transported for steroid hormone biosynthesis due to the increased overall cholesterol supply (and an increase in average deliverable cholesterol load per HDL particle), resulting in higher levels of steroids. The extra steroids in turn will suppress steroidogenesis via the negative feedback mechanism to protect the body from elevated steroid hormone levels, leading to the HDL-C reduction. This is consistent with the fact that higher LDL-C is mostly associated with higher LDL/HDL ratio. Although Western diet increases steroidogenesis and causes puberty age reduction, the effect is relatively small due to the steroid hormone homeostasis control, and most of the clinical data indicate that total cholesterol in circulation is not statistically correlated with steroid hormone levels (Mondul A, et al. 2010 Association of serum cholesterol and cholesterol-lowering drug use with serum steroid hormones in men in NHANES III. Cancer Causes Control 21:1575-1583). LDL-C can be moderately reduced through dietary control but the effect on HDL-C is variable, depending upon the extent and type of the dietary control since the HDL-C effect is a combination factor of dietary fat and steroid hormone homeostasis feedback. Polyunsaturated dietary fat raises both HDL-C and LDL-C (Maki K, et al. 2010 Baseline lipoprotein lipids and low-density lipoprotein cholesterol response to prescription omega-3 acid ethyl ester added to simvastatin therapy. Am. J. Cardiol. 105:1409-1412), and a typical low-fat diet reduces both LDL-C and HDL-C and, however, atheroprotective potential of the HDL (positively correlated to C_{HDL}) is improved despite reduction of HDL-C (Roberts C, et al. 2006 Effect of a short-term diet and exercise intervention on inflammatory/anti-inflammatory properties of HDL in overweight/obese men with cardiovascular risk factors. J. Appl. Physiol. 101:1727-1732). Apparently, the lower HDL-C is compensated by higher HDL capacity to maintain the steroid hormone homeostasis. Long-term calorie restriction significantly lowered sex steroid hormone levels in men (Cangemi R, et al. 2010 Long-term effects of calorie restriction on serum sex-hormone concentrations in men. Aging Cell 9:236-242), presumably due to the significantly lowered lipid levels.

Statins dramatically reduce LDL-C and triglycerides by blocking cholesterol biosynthesis, and significantly increase HDL-C as a class effect. The counterintuitive HDL effect can be explained as that in order to compensate for the cholesterol supply shortage caused by statins (and lowered average deliverable cholesterol load per HDL particle), HDL level would be upregulated to transport sufficient cholesterol to steroidogenic tissues to maintain steroid hormone homeostasis, although the dose response of different statins in HDL-C elevation are not equal (Yamashita S, et al. 2010 Molecular mechanisms of HDL-cholesterol elevation by statins and its effects on HDL functions. J. Atheroscler. Thromb. 17:436-451). The high percentage LDL-C reduction by statins would tip the balance of steroid hormones and this impact was observed in some clinical studies utilizing potent or higher dose statins. For example, in one study, the total T in diabetic men was lowered by statin therapy without significant effect on the homeostasis controlled by free T (Stanworth R D, et al. 2009 Statin therapy is associated with lower total but not bioavailable or free testosterone in men with type 2 diabetes. Diabetes Care 32:541-546).

People with a defective gene mutation at LDLR, apoB-100, PCSK9, or ARH have a LDL clearance defect that leads to LDL accumulation in blood. Since the higher LDL-C may not be necessarily the result of higher hepatic cholesterol level, familial hypercholesterolemia patients have normal or slightly lower HDL-C (Ikonen E. 2006 Mechanisms for cellular cholesterol transport: defects and human disease. Physiol. Rev. 86:1237-1261). By the SHAC1 mechanism, capacity of LDL in familial hypercholesterolemia patients should be reduced significantly due to the defect of efficient delivery of cholesterol, although the effect of LDL capacity variation on atherosclerosis has not been studied. It was reported that LDL subfractions in patients with familial disorders of lipoprotein metabolism were different from other patient groups (Berneis K K, Krauss R M. 2002 Metabolic origins and clinical significance of LDL heterogeneity. J. Lipid Res. 43:1363-1379).

Effect of Perturbation of Sex Steroid Hormone Homeostasis on HDL-C

HDL-C changes associated with sex steroid hormone perturbations can be also explained by the mechanism that HDL-C is positively correlated with endogenous sex steroid production to meet the supply and demand of cholesterol. Total testosterone has a positive correlation with HDL-C in healthy males (Agirbasli M, et al. 2010 Sex hormones, insulin resistance and high-density lipoprotein cholesterol levels in children. Horm. Res. Paediatr. 73:166-174; Nordoy A, et al. 1979 Sex hormones and high density lipoproteins in healthy males. Atherosclerosis 34:431-436). Age-related androgen decline in men is also associated with HDL-C reduction based on some prospective studies (Walter M. 2009 Interrelationships among HDL metabolism, aging, and atherosclerosis. Arterioscler. Thromb. Vasc. Biol. 29:1244-1250). Adult males with Klinefelter's syndrome have a low level of androgen, elevated LDL-C, and reduced HDL-C due to a genetic defect blocking androgen biosynthesis in spite of high circulating gonadotropins (Bojesen A, et al. 2006 The metabolic syndrome is frequent in Klinefelter's syndrome and is associated with abdominal obesity and hypogonadism. Diabetes Care 29:1591-1598).

The SHAC1 mechanism explains a long standing contradictory observation that exogenous and endogenous androgens behave differently in lipid modulation (Manolakou P, et al. 2009. The effects of endogenous and exogenous androgens on cardiovascular disease risk factors and progression. Reprod. Biol. Endocrinol. 7:44). An increase in endogenous androgen production would boost HDL-C to meet the biosynthesis cholesterol needs, while the use of exogenous androgens will reduce endogenous androgen production via suppression of LH. In most of the clinical studies, androgen therapy is associated with HDL reduction, especially at super-physiological doses. Healthy young adults have a perfect hormonal homeostasis and are the most sensitive to exogenous androgens (Hartgens F, et al. 2004 Effects of androgenic-anabolic steroids on apolipoproteins and lipoprotein (a). Br J Sports Med 2004; 38, 253-9). Most of high-dose anabolic steroid abuse is not reported in scientific literature, although the dramatic reduction in HDL has been observed by physicians. The exogenous androgen effect on HDL would be minimal when given at physiological level in aging-related hypogonadal population since their endogenous androgen production system is less efficient and less sensitive to perturbation.

Parenteral or topical administration of exogenous androgen may have less impact on HDL-C, which suggests that androgens may have a direct feedback mechanism through the liver. The caveat is that oral androgens are usually more potent than non-oral androgens and direct comparison of different administrations of the same androgen has not been done in humans.

Women normally have higher HDL-C than men and it is generally believed that estrogen raises HDL through direct regulation of HDL metabolism in the liver. It is not understood, however, why HDL-C is little changed at menopause when estrogen level is significantly reduced. Nonetheless, the SHAC1 mechanism explains why endogenous hormone production in women would consume more cholesterol than in men and thus requires more HDL to deliver more cholesterol to meet this demand. In the menstrual cycle, HDL-C almost synchronizes with female hormone changes (Mumford S, et al. 2010 A longitudinal study of serum lipoproteins in relation to endogenous reproductive hormones during the menstrual cycle: findings from the BioCycle study. JCEM 95:E80-E85). HDL-C in women at low hormone stage is much closer to HDL-C in men of similar age, which is consistent with the fact that E2 and progesterone levels at menses are only slightly higher than the female hormone levels in men. HDL-C in women increases significantly during pregnancy and synchronizes with the dramatic increase in progesterone production to support pregnancy (Mankuta D, et al. 2010 Lipid profile in consecutive pregnancies. Lipids Health Dis. 9:58). At menopause, E2 level normally drops more than 50% and HDL-C is only slightly decreased. E2 in circulation is a tiny fraction of DHEA sulfate (DHEAS), a precursor of estrogen biosynthesis, that does not drop as dramatically as E2 at menopause (Davison S, et al. 2005 Androgen levels in adult females: changes with age, menopause, and oophorectomy. JCEM 90:3847-3853), as a result, cholesterol need for steroid biosynthesis does not change much at menopause.

Sex steroid hormone and lipid levels are known to be different among different race/ethnic groups. Africans seems to have higher HDL-C (Harman J L, et al. 2011 Age is positively associated with high-density lipoprotein cholesterol among African Americans in cross-sectional analysis: The Jackson Heart Study. J. Clin. Lipidol. 5:173-178) and sex hormone levels than other groups (Rohrmann S et al. 2007 Serum estrogen, but not testosterone, levels differ between black and white men in a nationally representative sample of Americans. JCEM 92:2519-2525), which is in consistent with the mechanism that HDL-C is positively correlated with steroid hormone biosynthesis.

Exogenous progestins can inhibit endogenous sex steroid hormone production via suppression of LH, which would reduce HDL-C. Older generation oral progestins generated HDL-C reduction in women to a level similar as seen in men using anabolic steroids, which is partially the result of the androgenic cross-reactivity. Even the parenterally formulated progestin-only contraceptive, depot medroxyprogesterone acetate that has much less androgenic activity, showed a significant reduction in HDL-C (Berenson A, et al. 2009 Effects of injectable and oral contraceptives on serum lipids. Obstet. Gynecol. 114:786-794). Most clinical use of female hormones is a combination of progestin and estrogen. Due to the opposite lipid effect of estrogens from progestins, clinical results of lipid profile in hormone replacement therapy (HRT) and oral contraceptives (OC) are quite variable. Additionally there are many other variables that influence lipids, such as doses, routes of administration, pattern and timing of treatment, which makes it difficult to establish a relationship between lipid effect and a specific cardiovascular risk even with a large trial population (Turgeon J L, et al. 2004 Hormone therapy: Physiological complexity belies therapeutic simplicity. Science 304:1269-1273). In general, oral estrogens have been shown to raise HDL-C and there is no or minimal effect when estrogen is given transdermally. The results seem to contradict the SHAC1 mechanism but can be explained by the very unique dual (negative and positive) feedback mechanism of estrogens. At a low concentration, E2 suppresses LH/FSH in the early part of the follicular phase in a negative feedback loop and when a high concentration threshold is reached, E2 causes the LH/FSH surge before ovulation by turning the feedback mechanism positive. A rodent study suggests that the opposite dual feedback mechanisms are managed by the two estrogen subtype receptors expressed in the neurons (Hu L, et al. 2008 see above). When used as OC or HRT, estrogen is generally given at doses that generate circulating levels higher than the normal peaks in the menstrual cycle, and would trigger the positive feedback mechanism to stimulate more hormone production. It was reported that when postmenopausal women were given oral E2, the estrone level increased more than 10-fold (Vehkavaara S, et al. 2000 Differential effects of oral and transdermal estrogen replacement therapy on endothelial function in postmenopausal women. Circulation 102:2687-2693). Estrone is the immediate precursor of E2 biosynthesis and the elevation is compatible with the positive feedback mechanism that oral E2 boost endogenous estrogen production and HDL-C.

Estrogens have been used in men with prostate cancer intended to suppress endogenous T production and it turned out that the free T level was reduced through increase of SHBG instead of reduction in T production (Purnell J Q, et al. 2006 Effects of transdermal estrogen on levels of lipids, lipase activity, and inflammatory markers in men with prostate cancer. J. Lipid Res. 47:349-355). Men treated with synthetic oral estrogens for transsexual purpose were found to have increase LH/FSH and E2 levels (Sosa M, et al. 2004 Serum lipids and estrogen receptor gene polymorphisms in male-to-female transsexuals: effects of estrogen treatment. Eur. J. Internal Med. 15:231-237), which indicates that oral estrogens increase endogenous estrogen production in males. A recent result of high-dose isoflavone showed that HDL-C and total T were significantly reduced in postmenopausal women (Basaria S, et al. 2009 Effect of high-dose isoflavones on cognition, quality of life, androgens, and lipoprotein in post-menopausal women. J. Endocrinol. Invest. 32:150-155), which should be a case where only the negative feedback mechanism works due to the very low potency of the phytoestrogen.

### Effect of Corticosteroids on HDL-C

The corticosteroids are mainly synthesized in the adrenal gland, and their negative feedback control through HPA is much weaker than that of sex steroid hormones. Aldosterone in circulation is a tiny fraction of corticosteroids so that it is unlikely to have a meaningful correlation with HDL-C through its biosynthesis need. Aldosterone levels in primary aldosteronism patients are often correlated positively with hypertension and inversely with HDL-C (Funder J W, Reincke M. 2010 Aldosterone: a cardiovascular risk factor? Biochim. Biophys. Acta. 1802:1188-1192). Angiotensin II and potassium are the two major regulators of aldosterone homeostasis and thus the HDL-C effect is likely related to imbalance of the renin-angiotensin system. Nevertheless, the HDL-C reduction and aldosterone elevation are consistent in terms of their roles as cardiovascular disease risk factors. Cortisol is mainly a glucocorticoid and also plays a significant role similar to that of aldosterone due to its cross-reactivity on the mineralocorticoid receptor (MR) and being at a relatively high concentration. The plasma cortisol level has a unique diurnal pattern and shows significant variation among people. There is very limited literature data indicating the relationship between cortisol levels and HDL-C in healthy people. Increased cortisol level in early postmenopausal women is associated with insulin resistance and decreased HDL-C (Cagnacci A, et al. 2011 Increased cortisol level: a possible link between climacteric symptoms and cardiovascular risk factors Menopause 18:273-278). People with SR-BI mutations have significantly reduced corticosteroid production in comparison with control (Vergeer M, et al. 2011, see above), which can be viewed as equivalent of having low HDL-C and lower corticosteroid levels. Patients with congenital adrenal hyperplasia, a genetic disease in which corticosteroid biosynthesis is impaired by mutations in 21α-hydroxylase or 11β-hydroxylase, normally have higher LDL-C and lower HDL-C (Zimmermann A, et al. 2010 Alterations in lipid and carbohydrate metabolism in patients with cliassic congenital adrenal hyperplasia due to 21-hydroxylase deficiency. Horm. Res. Paediatr. 74:41-49), which is consistent with the hormone biosynthesis feedback mechanism. People with endogenous Cushing's syndrome tend to have elevated triglycerides and total cholesterol, and literature data on their HDL-C is mixed due to pooling data with several distinct pathophysiological causes.

Exogenous corticosteroids are known to be associated with dyslipidemia, metabolic syndromes, and HDL-C elevation in patients with inflammatory disease. A small study in healthy volunteers indicating HDL-C was elevated with little LDL-C change after short term dexamethasone treatment, which is partially attributed to the suppression of endogenous corticosteroids whose MR agonist activity is inversely correlated with HDL-C (Brotman D J, et al. 2005 Effects of short-term glucocorticoids on cardiovascular biomarkers. JCEM 90:3202-3208). In a retroanalysis of clinical records of systemic lupus erythematosus patients, corticosteroid treatment significantly increased CHD risk despite of the increase of HDL-C (Karp I, et al. 2008 Recent corticosteroid use and recent disease activity: Independent determinants of coronary heart disease risk factors in systemic lupus erythematosus? Arthritis Rheum. 59:169-175), which suggests that synthetic corticosteroid treatment compromises HDL capacity probably due to metabolic syndrome related mechanisms.

### Complementary Feedback Mechanism Through the Liver

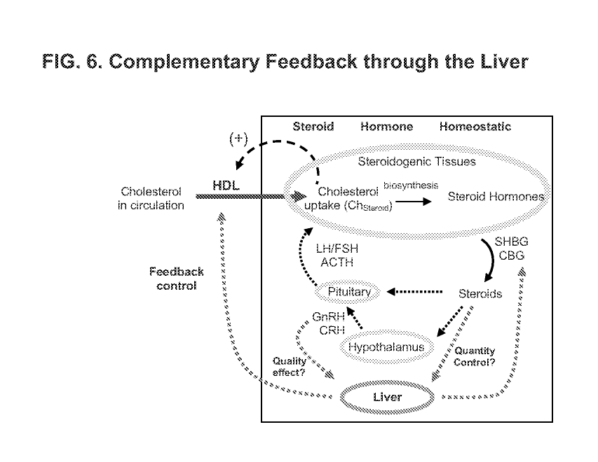

Although molecular details of the global control of HDL homeostasis by cholesterol uptake for steroidogenesis are not known, the control has to go through the liver that manages metabolism of both cholesterol and lipoproteins. It has been demonstrated that orally administered sex steroid compounds have effect on HDL-C significantly bigger than that of the compounds given non-orally due to higher first-pass liver exposure of an oral compound. The amplified HDL-C effect indicates that feedback through the liver may be the key of controlling cholesterol delivery for steroidogenesis, which is complementary to the HPG/HPA feedback in controlling steroid hormone biosynthesis in steroidogenic tissues (FIG. 6). Endogenous steroid hormones are produced in steroidogenic tissues so that compound exposure levels in hypothalamic-pituitary glands and in the liver should be similar. When an oral androgen is given, androgen concentration goes up in the liver much more than in circulation. As a result, HDL-C and endogenous T could be lowered without significant change in LH. When an oral estrogen is given, HDL-C and endogenous E2 could be increased without significant changes in gonadotropins.

GnRH modulators have been used to knock down endogenous androgen production (chemical castration) by suppressing LH in healthy and prostate disease patients, and demonstrated a clear elevation of HDL-C (Bhasin S, et al. 2001 Testosterone dose-response relationships in healthy young men. Am J Physiol Endocrinol Metab 281:E1172-E1181; Saylor P J, Smith M R. 2009 Metabolic complications of androgen deprivation therapy for prostate cancer. J. Urol. 181:1998-2008). Since the T production is shut down by GnRH modulators, elevated HDL-C means compromised HDL capacity by the modulators based on equation (1). GnRH analogs or agonists generate pituitary exposure at a supraphysiological level and lead to pituitary shut down so that the agonistic activity becomes strong antagonistic. GnRH analogs or modulators also have liver exposure at a supraphysiological level, which may directly affect lipoprotein homeostasis. LDL-C and triglycerides are also increased along with HDL-C in patients treated with a GnRH compound. It has been reported that statins totally lost lipid lowering efficacy in prostate cancer patients who were treated with a GnRH agonist or antagonist (Yannucci J, et al. 2006 The effect of androgen deprivation therapy on fasting serum lipid and glucose parameters J. Urol. 176:520-525). This indicates that GnRH modulators would temper lipoprotein capacity of cholesterol transportation including LDL, and thus LDL-C had little change in spite of lowered cholesterol biosynthesis by statins. It seems that GnRH compound level in the liver influences capacity of lipoproteins in cholesterol transportation.

GnRH analog or modulator treatment is equivalent of high androgen exposure in hypothalamus-pituitary glands and low androgen level in the liver, which can be viewed as an opposite scenario of administration of oral androgens that have much higher liver exposure than that in circulation. Since androgen effect in the liver is androgen receptor mediated and higher than physiological level of androgens in the liver causes HDL-C reduction, it is reasonable to believe that androgen in the liver below the physiological level would cause HDL-C to raise. In subjects treated with a GnRH compound, the resulted HDL-C is the result of a high GnRH compound level and a low androgen level in the liver. Surgical castration in men did not affect HDL-C significantly and raised triglycerides and LDL-C gradually (Xu T, et al. 2002 Effect of surgical castration on risk factors for arteriosclerosis of patients with prostate cancer. Chin. Med. J. (Engl.) 115:1336-1340). The difference between the castrations is that the liver exposure of LH and GnRH levels is significantly different. Since no cholesterol is delivered for steroidogenesis in testes in the surgical patients, cholesterol in circulation is increased as indicated by the increase of triglycerides and LDL-C. The seemingly unchanged HDL-C is the collective result of a lower androgen level in the liver to drive HDL higher and the lower cholesterol needs for steroidogenesis to drive HDL lower. In surgical menopause women, HDL-C was slightly decreased with increases in triglycerides and LDL-C (Tuna V, et al. 2010 Variations in blood lipid profile, thrombotic system, arterial elasticity and psychosexual parameters in the cases of surgical and natural menopause. Aust. N. Z. J. Obstet. Gynaecol. 50:194-199). It seems that steroid hormone levels in the liver are correlated with HDL quantity.

Steroid binding globulins, SHBG and CBG, are vehicles to transport steroids in circulation and are mainly produced in the liver. It has been shown in many different settings that SHBG is positively correlated with HDL-C, which suggests that SHBG is correlated with endogenous sex hormone production and is part of the feedback loop controlled by the liver. Steroid binding globulins can be viewed as buffer systems to maintain endogenous steroid hormone homeostasis. When E2 biosynthesis is increased by oral estrogens, SHBG level increases to counteract rising free E2 level, and when T production decreases by oral androgens, SHBG decreases to compensate the free T reduction. GnRH treatment gives a mixed signal in the liver where hormone and LH levels point to opposite directions of endogenous hormone production, which is compatible with the observation that SHBG level did not change in men by GnRH treatment (Bhasin S, et al. 2001, see above) or surgical castration (Xu T, et al. 2002, see above). SHBG is increased with aging in men (Muller M, et al. 2003 Endogenous sex hormones in men aged 40-80 years. Eur. J. Endocrinol. 149:583-589) and the long term effect is associated with overall hormone reduction during aging process, which is a role different from buffering daily hormonal variations.

### Underlying Relationships Among CHD Risk Factors

American Heart Association currently lists the following CHD risk factors: age, gender, heredity, smoking, cholesterol, blood pressure, physical inactivity, obesity, and diabetes mellitus. The cause of CHD is cellular cholesterol trafficking induced atherosclerosis so that both LDL-C and HDL-C are the major factors. Smoking and blood pressure are more or less triggers of CHD, and heredity reflects genetic weakness in other risk factors. Age, gender, physical inactivity, obesity, and diabetes are all related to steroid hormones, and can be linked with cholesterol trafficking by the SHAC1 homeostatic system. Based on the mechanism, relationships of CHD risk, LDL-C, HDL-C, C_{HDL}, and Ch_{Steroid} can be expressed mathematically as

CHD Risk=f{LDL-C/(HDL-C×C_{HDL})}=f{LDL-C/Ch_{Steroid}} (3)

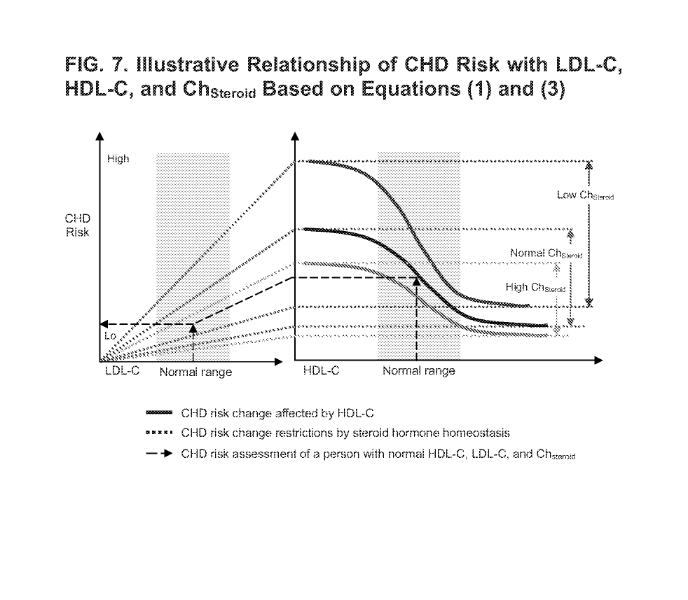

The cholesterol uptake amount from circulation for steroid hormone biosynthesis is a significant new factor in assessing CHD risk. Equation (3) can be schematically illustrated in FIG. 7 and qualitatively used to assess CHD risk based on blood cholesterol and steroid hormone levels. For example, it was reported that HDL-C was disassociated from CHD risk in patients with very low LDL-C after statin treatment in multiple large clinical trials (Ridker P M, et al. 2010 HDL cholesterol and residual risk of first cardiovascular events after treatment with potent statin therapy: an analysis from the JUPITER trial. Lancet 376:333-339 and 1738-1739). By equation (3)/FIG. 7, the impact of the HDL variation (ΔHDL-C) on CHD risk will gradually diminish along with the LDL-C reduction and, as a result, detection of clinical significance of the HDL variation would be more challenging when LDL-C is low. It was reported separately that a subgroup of people with lower HDL-C due to genetic mutations in ABCA1 did not have the expected higher risk of ischemic heart disease (Frikke-Schmidt R, et al. 2008 Association of loss-of-function mutations in the ABCA1 gene with high-density lipoprotein cholesterol levels and risk of ischemic heart disease. JAMA 299:2524-2532). Equation (1) indicates that higher HDL capacity would compensate for the transportation “vehicle” shortage to maintain the steroid hormone balance. Equation (3)/FIG. 7 also explain why directly targeting HDL-C as a drug therapy being less effective. Since the strict endocrine control would lower the HDL capacity significantly to protect the body from high levels of steroid hormones, CETP inhibitors have difficulty in clinic to demonstrate robust efficacy in reducing cardiovascular events despite >100% HDL-C elevation (Cannon C P, et al. 2010 Safety of anacetrapib in patients with or at high risk for coronary heart disease. N. Engl. J. Med. 363:2406-2415). Based on the same mechanism, it is not surprise that adding high dose niacin on top of statin therapy to raise HDL-C showed no significant reduction in cardiovascular events (National Heart, Lung, and Blood Institute. 2011 NIH stops clinical trial on combination cholesterol treatment. NIH News Release May 26).

In general, the linear relationship of HDL-C and CHD risk is plateaued outside the normal HDL-C range due to the restriction of steroid hormone homeostatic factor Ch_{Steroid}. The plateau effect is achieved by the interchange of HDL-C and its capacity of cholesterol transportation described in equation (1). The physiological consequence of the effect is that ultrahigh HDL-C would not provide the ultrahigh benefit and ultralow HDL-C would not mean ultrahigh risk if steroid hormone homeostasis is balanced. As indicated in FIG. 7, CHD risk is lowest when Ch_{Steroid} and HDL-C are both higher and LDL-C is lower.

### Cholesterol and Venous Thrombosis Risk Factors

Oral estrogen plays an important role in RCT to reduce cholesterol level in circulation via increase of HDL-C and, as a result, cholesterol concentration in the bile is significantly increased after estrogen treatment, which led to higher frequency of gallstone disease even in men (Henriksson P, et al. 1989 Estrogen-induced gallstone formation in males. J. Clin. Invest. 84:811-816). Venous thrombosis (VT) seems to be associated with estrogen treatment and the molecular mechanism has not been established. The SHAC1 mechanism may help to understand the relationship between VT and estrogens.

It has been known that plasma cholesterol level has noticeable effects on many hematological parameters. In SR-BI-knockout mice (Dole V S, et al. 2008 Thrombocytopenia and platelet abnormalities in HDL receptor-deficient mice. Aeterioscler. Thromb. Vasc. Biol. 28:1111-1116) and in humans with SR-BI mutation (Vergeer M, et al. 2011, see above), cholesterol content in platelets is significantly increased with reduced platelet aggregation. The increase of cholesterol in platelets is partially due to the increase of overall plasma cholesterol level and partially the result of insufficient exchange of cholesterol with lipoproteins via SR-BI. Surgical menopausal women also have prolonged bleeding/clotting time along with increases of LDL-C and VLDL-C, and slight decrease of HDL-C (Tuna V, et al. 2010, see above), and bilateral salpingo-oophorectomy in some extend is similar as SR-BI deficiency since in both cases HDL capacity in cholesterol delivery is compromised, which is consistent with equation (1) that C_{HDL} should be reduced after oophorectomy due to reduction in Ch_{Steroids} when HDL-C remains same or slightly reduced. The thrombosis increase caused by estrogens is compatible to the SHAC1 mechanism where estrogens increase HDL productivity in cholesterol disposal and thus reduce cholesterol content in circulation as well as in platelets, and thus enhance platelet aggregation. Apparently, the estrogen effect on VT is quite small in clinical observations and women with more VT risk factors such as hereditary weakness in coagulant factors, older age, smoking, obesity, and immobilization are most vulnerable to have VT with estrogen treatment. Based on the SHAC1 mechanism, VT risk of estrogens is associated with their atheroprotective activity via RCT, although VT may cause cardiovascular events and stroke in people who have already developed atherosclerosis. The higher VT risk disappeared after stopping the estrogen therapy in WHI trial patients who had hysterectomy (LaCroix A Z, et al. 2011 Health outcomes after stopping conjugated equine estrogens among postmenopausal women with prior hysterectomy: a randomized controlled trial. JAMA 305:1305-1314). Hysterectomy is frequently done together with oophorectomy. Based on the SHAC1 mechanism, the atheroprotective effect of estrogens could be blunted since the higher HDL-C raised by estrogens can not increase more cholesterol consumption for endogenous hormone production, which may explain the WHI trial result that there was no CHD difference between the estrogen-only and placebo groups. A number of publications described that esterified estrogens (EE) did not have any treatment related VT risk (Smith N L, et al. 2004 Esterified estrogens and conjugated equine estrogens and the risk of VT. JAMA 292:1581-1587) and, however, EE treatment increase neither HDL-C nor SHBG as typical estrogens do (Chiuve S E, et al. 2004 Effect of the combination of methyltestosterone and esterified estrogens compared with esterified estrogens alone on ApoCIII and other apolipoproteins in VLDL, LDL, and HDL in surgical postmenopausal women. JCEM 89:2207-2213).

HDL capacity, C_{HDL}, is a determinant of atherosclerosis and can be affected by genetic defects, pathophysiological states, or medical interventions. Currently, there is no standard procedure to measure or estimate C_{HDL}. Based on equations (1) and (3), C_{HDL} can be replaced with HDL-C and Ch_{Steroid} that can be estimated by circulating overall steroid levels. A quantitative formula to assess CHD risks can be developed by analyzing clinical data, and more convenient test kids can be developed to collecting the relevant data such as LDL-C, HDL-C, total testosterone, total estrogen and progesterone, and DHEAS as a novel method to detect or measure potential risks of atherosclerosis in general population. Alternatively, genetic testing and data analysis of the molecular networks related to the SHAC1 homeostatic system can be developed based on the mechanism to better assess or predict CHD or atherosclerosis risks in humans.

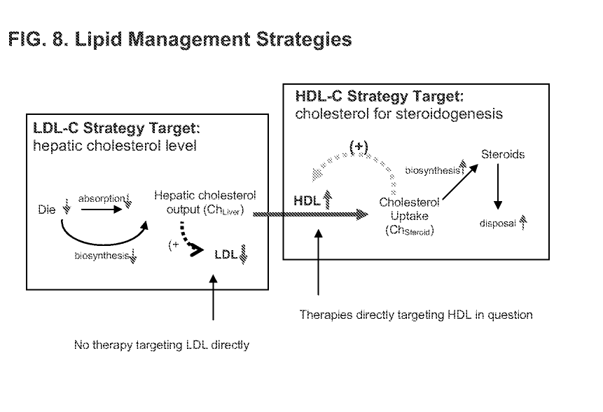

Lipoprotein metabolism consists of multiple complex molecular networks that still have many details to be learned. It has been demonstrated that lowing LDL-C and triglycerides significantly reduced atherosclerosis and cardiovascular events, and the most successful strategy of lowing lipids is the one targeting the sources that controls LDL-C as well as triglycerides (FIG. 8). In comparison with molecular details of lipoprotein metabolism, hepatic cholesterol pathways are much more known and the level can be controlled through reduction in dietary intake, biosynthesis, and absorption at enterocytes. Reduction of cardiovascular risks in addition to lowering LDL-C has been current focus of drug discovery and development in lipid field. Directly targeting HDL-C has made progress and however shown to be difficult to demonstrate clinical benefit of reducing cardiovascular events. The present invention describes a much better alternative of targeting the cholesterol uptake for steroidogenesis, Ch_{Steroid}, that controls HDL productivity in cholesterol transportation, both quantity and capacity (FIG. 8). By increasing endogenous sterol biosynthesis, cholesterol uptake from circulation will be increased, which will drive up needs for increased either HDL quantity or capacity, or both. As a result, higher productivity of HDL in RCT will further reduce or prevent atherosclerosis. Increase in endogenous steroid hormone production does not necessarily increase free hormone levels since the steroid binding proteins will change accordingly to maintain the steroid hormone homeostasis, which can be an advantage of the strategy.

Some embodiments include methods of treating a disorder or condition associated with the balance of the SHAC1 homeostatic system in a patient in need of such treatment. Some methods include administering an initial effective amount of a regiment that is developed based on control of the SHAC1 homeostatic system.

In some embodiments, the disorder or condition is associated with dyslipidemia, dyscholesterolemia, dyslipoproteinemia, and/or atherosclerosis or cardiovascular events. Reduction of CHD or atherosclerosis risk can be achieved by adjusting balance of the system with multiple methods of choices. There are multiple enzymes, cofactors, and intermediates involved in steroid hormone biosynthesis pathways, and the hormonal homeostasis is controlled by the steroid hormone receptors and steroid binding globulins.

In some embodiments, the method of increasing cholesterol consumption for steroidogenesis is related to targeting an enzyme to facilitate one or more biotransformation processes. In some embodiments, the method of increasing cholesterol consumption for steroidogenesis is related to targeting a cofactor that can facilitate one or more biosynthetic steps. In some embodiments, the method of increasing cholesterol consumption for steroidogenesis is related to targeting an intermediate that can be accumulated or metabolized to one or more species that is(are) outside the homeostatic pathways. In some embodiments, the method of increasing cholesterol consumption for steroidogenesis is related to targeting a receptor that can modulate steroid production. In some embodiments, the method of increasing cholesterol consumption for steroidogenesis is related to targeting a binding protein that can modulate bioactive concentration of one or more steroid hormones.

Some embodiments include methods of intervening steroid hormone balance to achieve a medical purpose in humans. Such methods include administering an effective amount of a non-peptidyl small molecule, a peptide, a biologic molecule, an antibody, an antisense molecule, a small interfering RNA molecule, a gene therapy, or stem cell therapy that has intended hormonal effects with increasing or without decreasing cholesterol consumption for sterol biosynthesis, and/or that does not increase VT risk.

In some embodiments, the methods are related to new generations of steroid hormone receptor modulators that do not cause any negative lipid effects by increasing or without decreasing in cholesterol consumption for sterol biosynthesis. In some embodiments, the modulators are selective androgen receptor modulators (SARMs) that do not negatively affect endogenous hormone production. In some embodiments, the modulators are selective progesterone receptor modulators (SPRMs) that do not negatively affect endogenous hormone production. In some embodiments, the modulators are selective estrogen receptor modulators (SERMs) that do not negatively affect endogenous hormone production and VT risk factors. In some embodiments, the modulators are selective glucocorticoid receptor modulators (SGRMs) that do not negatively affect endogenous hormone production.

In some embodiments, the methods are related to new generations of steroid hormone regiments that do not cause any negative lipid and/or VT effects by selectively mixing two or more hormonal compounds with opposite lipid profiles.

The following examples are set forth merely to assist in understanding the embodiments and should not be construed as limiting the embodiments described and claimed herein in any way. Variations of the invention, including the substitution of all equivalents now known or later developed, which would be within the purview of those skilled in the art, and changes in formulation or minor changes in experimental design, are to be considered to fall within the scope of the invention incorporated herein.

### Example I

### New Generation SARM Compounds with Minimal or No Negative Lipid Effect

Steroidal androgens have been used to treat a variety of male disorders such as hypogonadism. A number of SARMs have been investigated for the treatment of musculoskeletal disorders, such as bone disease, muscle wasting disease, and age-related frailty, and for hormone replacement therapy (HRT), such as female androgen deficiency. It has been demonstrated that in preclinical animal models SARM compounds have a favorable tissue selective profile of maintaining full activities in muscle, bone, and CNS, and significantly reduced activities in prostate and sebaceous glands (Vajda E G, et al. 2009 Pharmacokinetics and pharmacodynamics of LGD-3303, an orally available nonsteroidal-selective androgen receptor modulator). Since the unknown nature of the complex relationships of lipid profile and androgens, there has been no preclinical model available to address the concerns about potential negative effect of lipid profile of androgens, especially the long-term effect of androgens on cardiovascular system. As a result, development of SARMs faces unpredictable regulatory and scientific challenges despite the overwhelming efficacy of the modulators and indisputable unmet medical needs.

A novel mechanism is described that offers a solution to the outstanding issue. Based on the mechanism, exogenous androgens reduce endogenous T production by suppression of LH via the HPG axis and by suppression of HDL via the liver. The HDL effect can be significantly exaggerated if an androgen is given orally due to the first-pass liver exposure that is much higher relative to that of HPG axis. Similar to other nuclear receptor ligand, androgens play their genomic roles in a very tissue-selective fashion, which occurs naturally through tissue-selective expression of androgen receptor (AR) and many other related genes. When an androgen binds to AR and causes the receptor protein to adopt a ligand-specific conformation, the complex needs to recruit other genes (cofactors) to achieve transcriptional changes in a tissue-selective or tissue-specific setting. The mix of related genes attenuates androgen activity in a specific tissue. It has been demonstrated that more tissue-selectivity of SARM compounds can be developed to separate anabolic and androgenic activities of T by optimizing compounds based on in vitro assays with tissue-selective genes of choice. In a similar fashion, new assays and models can be developed to dial down negative lipid profile of SARMs by minimizing their effect on endogenous hormone production. Specifically, establishment of a tissue-selectivity to spare the feedback loops in HPG and the liver will result in new generation SARMs that have minimal or no negative lipid effects.

Steroidal androgens and SARM compounds have been described in the literature with the target profile of maintaining anabolic activity and minimizing androgenic activity. Screening of the known compounds based on the new assays/models to characterize AR modulating activity in the liver and hypothalamic-pituitary glands should generate lead compounds of new generation of SARMs for further optimization. Some embodiments of the present invention include compounds that have tissue-selective AR modulating activities to maintaining anabolic activity in bone, muscle, and CNS, minimizing androgenic activity in prostate and skin, and reducing the HDL-lowering effect.

### Example II

### AR Modulating Compounds with HDL Productivity Enhancement Activity

AR antagonists are used to treat prostate diseases by reducing or eliminating AR mediated transcriptional activation via competitive binding to AR with endogenous androgens. Anti-androgens are known to elevate LH levels that in turn increase steroid hormone biosynthesis including T (Eri L M, et al. 1995 Effects on the endocrine system of long-term treatment with the non-steroidal anti-androgen Casodex in patients with benign prostatic hyperplasia. Br. J. Urol. 75:335-340). As a result, this would lead to an increase in HDL productivity in transporting cholesterol. As described in Example I, a tissue-selective AR modulator can be developed based on the new assays/models to characterize AR modulating activity in the liver and hypothalamic-pituitary glands. The modulator compounds have AR antagonistic activity in the liver and/or hypothalamic-pituitary glands, and maintain AR agonistic or partial activity in bone and muscle, or have reduced AR antagonist activity in muscle and bone. In other words, the compounds can selectively increase endogenous androgen production such as done by an AR antagonist and can not effectively compete with T in muscle and bone cells. Alternatively, a liver-targeting AR antagonist can be developed with the target profile of higher liver exposure to stimulate endogenous androgen and SHBG production, and of lower exposure in circulation to minimizing competitive binding to AR outside the liver.

Steroidal and nonsteroidal SARM compounds have been described in the literature have a range of AR antagonistic activities with some tissue-selectivity. Screening of the known compounds based on the new assays/models to characterize the AR modulating activity in the liver and hypothalamic-pituitary glands should generate lead compounds for further optimization to improve efficiency in HDL productivity enhancement activity. Some embodiments of the present invention include compounds that have tissue-selective AR modulating activities to have HDL productivity enhancement activity through the liver and/or hypothalamic-pituitary glands.

### Example III

### New Generation SPRM Compounds with Minimal or No Negative Lipid and/or Venous Thrombosis Effects

Progestins are widely used in OC and HRT in combination with estrogen and have a lipid profile very much similar to androgens. Due to the opposite lipid effect of progestins and estrogens, the negative lipid effect of progestins are often masked by estrogens, and the potential VT and cardioprotective effects of estrogens are often masked by progestins. Additionally, many progestins in the market have cross-reactivity with other steroid hormone receptors, which add another layer of complexity of the lipid effect. Venous thrombosis is a disorder associated with HRT in postmenopausal women and with OC in premenopausal women, and is distinct from the cardioprotective effect of estrogens through reduction of atherogenic risk factors. Medroxyprogesterone acetate, a synthetic progestin, doubled the thrombosis events in the large WHI trials (Cushman M, et al. 2004 Estrogen plus progestin and risk of venous thrombosis. JAMA 292:1573-1580). Separation of lipid effect from progestional effect has not been possible due to lack of understanding of the mechanism. Disassociation of the negative lipid effect of progestins from the beneficial effects will significantly reduce side-effects of progestin containing therapies and generate more clinical use in treatment of hormonal disorders or diseases.

Steroidal and nonsteroidal SPRM compounds are known to reduce the stimulative effect on breast tissues and maintain anti-estrogenic activity in uterus. Similar to the method of Example I, new assays and models can be developed based on the mechanism that reduction of negative feedback through the liver and hypothalamic-pituitary glands would lead to reduction of the HDL-lowering effect. Screening of the known compounds based on the new assays/models to characterize the PR modulating activity in the tissues of interest should generate lead compounds of new generation SPRMs that have reduced perturbation of the endogenous progesterone production and the desirable tissue-selectivity. Some embodiments of the present invention include compounds that have desirable tissue-selective PR modulating activities with neutral or reduced HDL-lowering activity.

### Example IV

### New Generation SERM Compounds with Reduced Venous Thrombosis Effect

Estrogens are still widely used in OC and are not equal in their VT effect. Due to the unknown mechanism of the effect, there has been no model to optimize estrogen compounds for the purpose. In the newer generation OC, much lower dose of estrogens has been used to reduce side effects including VT risk (Nelson A. 2010 New low-dose, extended-cycle pills with levonorgestrel and ethinyl estradiol: an evolutionary step in birth control. Int. J. Women's Health 2:99-106). Estrogen use in HRT has been mainly in short term treatment of menopausal symptoms since the early WHI trial conclusion in 2002 and 2004. SERM compounds have been developed with different profile from estrogens and demonstrated efficacy in prevention of osteoporosis and breast cancer in postmenopausal women without stimulation in uterus. Tissue-selectivity of SERMs is well characterized in bone, uterus, breasts, and CNS, and however has not been fully understood in lipids, hormones, cardiovascular, and VT risks. Clinical cardiovascular outcomes of different SERMs are different due to their selectivity profile differences and, however, VT risk of SERMs remains similar to that of estrogens. Although SERMs are partial or antagonistic at receptor level, they remain some agonistic activity in lipid modulation. Based on equation (3), the cardiovascular and VT events of SERMs would be complicated with the current clinical trial design where the patients were mixed with high percentage of lipid-lowering agent usage, hysterectomy with complete or partial oophorectomy that affect cholesterol and lipoprotein homeostasis.

As do estrogens, SERMs increase LH/FSH and SHBG in men and, thus, increase endogenous T production (Birzniece V, et al. 2010 Neuroendocrine regulation of growth hormone and androgen axes by SERMs in healthy men. JCEM 95:5443-5448). To take advantage of this effect on T production, others have proposed using SERMs to treat male sexual dysfunction, optionally in combination with a cGMP PDE5 inhibitor (Lee A G, et al. 2003 Methods of treatment for premature ejaculation in a male. U.S. Pat. No. 6,512,002). A SERM compound with reduced VT risk should result in more clinical use in treatment for sex hormone related disorders or conditions.

Steroidal and nonsteroidal SERM compounds have been described in the literature to have antagonistic activity in breasts and uterus and to maintain agonist activity in bone. Similar to the method of Example I, new assays and models can be developed based on the mechanism described in the present invention to characterize compound feedback profile through the liver and hypothalamic-pituitary glands. Screening of the known compounds based on the new assays/models to characterize the ER modulating activity in the tissues of interest should generate lead compounds of new generation SERMs that have desirable tissue-selectivity profile to meet a typical need. Some embodiments of the present invention include compounds that have ER modulating activities with neutral on lipid profile for contraceptive use without significant VT risk. Some embodiments of the present invention include compounds that have selective ER modulating activities with minimal effect on cholesterol content in platelets to reduce VT risk. Some embodiments of the present invention include compounds that have liver-targeting ER modulating activities with minimal level in circulation for prevention or treatment of atherosclerosis by increasing endogenous hormone and SHBG production with minimal or no VT risk. Some embodiments of the present invention include compounds that have liver-targeting ER modulating activities for treatment of sexual dysfunction with minimal or no VT risk.

### Example V

### New Generation of Contraceptive Regiment without VT Risk

Most widely used contraceptive agents are in combination of estrogen and progestin and are always associated with increase of VT events. Due to the mixed nature of the regiments and lack of understanding of the lipid-hormone-VT relationships, attempts to develop safer regiments or to explain the clinical result have not been very successful other than lowering the doses. It was observed that progestins in the combo regiment could affect estrogenic activity through antiestrogenic activity of some progestins and, as a result, the total estrogenicity of contraceptives concept was introduced to assess the associated VT risk (Tchaikovski S N and Rosing J. 2010 Mechanisms of estrogen-induced venous thromboembolism. Thromb. Res. 126:5-11). Attempts of lowering the VT risk by developing non-oral regiments have also failed (Cole J A, et al. 2007 Venous thromboembolism, myocardial infarction, and stroke among transdermal contraceptive system users. Obstet. Gynecol. 109:339-346).

Based on the SHAC1 mechanism of the present invention, exogenous estrogens increase HDL-C and decrease LDL-C by enhancing endogenous estrogen production and cholesterol consumption and bile excretion, which lead to the atheroprotective activity associated with VT risk, while exogenous progestins/androgens decrease HDL-C and increase LDL-C by suppression of endogenous hormone production. Taking advantage of the opposite lipid effects of estrogens and progestins/androgen, a new regiment can be developed to achieve neutral lipid effect without VT risk by selectively mixing estrogen(s) and progestin(s)/androgen.

Steroidal and non-steroidal estrogens and progestins (often associated with androgenic activity) have been described in literature to have different biological including lipid profiles. Screening known compounds using assays or models developed based on the SHAC1 mechanism should generate a combination of estrogen and progestin (with certain androgenic activity) with intended biological activity for contraception and neutralized lipid effects by cross compensation. The new regiment can be oral or optionally non-oral such as transdermal, vaginal ring, or depot injection.

### Example VI

### Compounds with SHBG Modulating Activity

SHBG has been used as a marker and never as a drug discovery target. SHBG is positively associated with HDL-C, sex steroid production, and other health factors such as insulin sensitivity (Peter A, et al. 2010 Relationships of circulating sex hormone-binding globulin with metabolic traits in humans. Diabetes 59:3167-31673) and body fitness (Morisset A S, et al. 2008 Impact of diet and adiposity on circulating levels of sex hormone-binding globulin and androgens. Nutr. Rev. 66:506-516). However, the cause and result relationships between SHBG and the factors have not been clearly described in the literature. Plasma SHBG level is controlled through its metabolism in the liver and can be affected by sex steroid receptor modulators and other nuclear receptor ligands. Based on the present invention, SHBG is part of the sex steroid hormone feedback mechanism through the liver, and increase in SHBG level either directly or indirectly could result in increases in endogenous steroid hormone production without increases of free steroid hormone levels and thus increasing HDL overall efficiency in cholesterol transportation and RCT to reduce atherosclerosis and increasing insulin sensitivity.

Sex steroid receptor modulators have been described in the literature to have a direct effect on SHBG level. Many orphan nuclear receptor ligands have been also known to affect SHBG level such as thyroid hormones and PPAR modulators. Screening the known compounds using assays or models developed based on the SHAC1 mechanism should generate lead compounds that can selectively increase SHBG level for further optimization to have therapeutic benefits in patients. Some embodiments of the present invention include compounds that have liver-targeting SHBG modulating activities through nuclear receptors with minimal level in circulation for treatment of lipid disorders, atherosclerosis, or diabetes/obesity.

SHBG contains several cation-binding sites in addition to the steroid hormone binding site (Avyakumov G V, et al. 2010 Structural analyses of sex hormone-binding globulin reveal novel ligands and function. Mol. Cell. Endocrinol. 316:13-23). Compound screening in a binding assay may generate lead compounds that can enhance SHBG binding affinity to steroids by binding to an allosteric binding site(s), which may provide an alternative method of increasing endogenous sex steroid production without necessarily increase of SHBG level. Some embodiments of the present invention include compounds that increase either SHBG level or binding affinity by other direct or indirect mechanisms for a therapeutic use such as upper-stream gene regulation or inhibition of SHBG catabolism.

### Example VII

### Molecules with Enhancement Activity of Endogenous DHEA Production

DHEA is a synthetic intermediate of several sex steroid hormones and mainly generated in adrenal cortex, and has been reported to have some broad but weak biological activities. DHEA is available in many countries as a dietary supplement due to its benign activity. In several observatory clinical studies, lower DHEA level in men was found to be casually associated with shorter lifespan (Enomoto M, et al. 2008 Serum DHEA sulfate levels predict longevity in men: 27-year follow-up study in a community-based cohort (Tanushimaru Study). J. Amer. Geriat. Soc. 56:994-998) and higher cardiovascular disease risks (Fukui M, et al. 2005 Serum DHEA sulfate concentration and carotid atherosclerosis in men with type 2 diabetes. Atherosclerosis 181:339-344). It is also known that regular exercise and calorie restriction increase DHEA level. However, DHEA supplements have not been demonstrated in clinic to have beneficial effects such as that of sex steroid hormones (Davis S R, et al. 2011 Clinical review: DHEA replacement for postmenopausal women. JCEM 96:1642-1653), although it is a prohibited substance in sports. As one of the intermediates of steroid biosynthesis, DHEA is one of the indicators of healthy level of endogenous steroid hormone production, based on the SHAC1 mechanism of the present invention, DHEA level should be associated with degree of health in lipid and metabolic profiles through the production of steroid hormones. As a result, molecules that enhance endogenous DHEA/DHEAS production (artificial adrenarch) should increase overall cholesterol consumption for steroid hormone synthesis and then have clinical benefits for the treatment of disorders or conditions related to atherosclerosis, cardiovascular, metabolic, and steroid hormones.

DHEA production depends on 17,20-lyase activity that is regulated by P450-oxidoreductase (POR), cytochrome b5 (CYB5A), and serine phosphorylation of P450c17 (CYP17A1) by a protein kinase (Pandey A V and Miller W L. 2005 Regulation of 17,20 lyase activity by cytochrome b5 and by serine phosphorylation of P450c17. J. Bio. Chem. 280:13265-13271). Small molecule inhibitors of 17,20 lyase have been in development for the treatment of prostate cancer. Screening the known compounds should be able to generate lead compounds that enhance the enzyme activity directly. Biologically engineered biologics with activity of POR or CYB5A can be developed for therapeutic uses to indirectly enhance the enzyme activity. Additionally, the enhancement can be also achieved by promoting the protein kinase activity via a therapeutically useful small molecule.

Majority of the DHEA in circulation is in form of DHEAS that is generated by sulfotransferase (SULT) enzymes and not available for steroid synthesis. Selective activation of the enzymes to increase non-active DHEAS is another strategy to increase endogenous DHEA production through the feedback mechanism. DHEAS can be converted back to DHEA by steroid sulfatase and has been considered as a steroid biosynthesis intermediate reservoir. The benign results of DHEA supplements and biologically non-active nature of DHEAS provide an opportunity to increase cholesterol consumption for steroid biosynthesis without significantly affecting overall steroid hormonal homeostasis.

### Example VIII

### Oral Hormone Replacement Therapies Combining a SARM and a SERM

Based on the SHAC1 mechanism of the present invention, exogenous estrogens increase HDL-C and decrease LDL-C by enhancing endogenous estrogen production and cholesterol consumption and bile excretion, which lead to the atheroprotective activity associated with VT risk, while exogenous androgens decrease HDL-C by suppression of endogenous T production. SERMs have been demonstrated to have atheroprotective activity and VT risks similar to that of estrogens, and SARMs have been shown to have lipid effects similar to androgens. Taking advantage of the opposite lipid effects of estrogens and androgens, a new SERM regiment with reduced VT risks, or a new SARM regiment with neutral lipid effect can be developed by selectively mixing a SERM and a SARM. In certain circumstances, a new SARM regiment can be developed by mixing with an estrogen, and a new SERM regiment can be developed by mixing with an androgen.

Steroidal and non-steroidal estrogens, androgens, SERMs, and SARMs have been described in literature to have different biological including lipid profiles. Screening known compounds using assays or models developed based on the SHAC1 mechanism should generate a combination of estrogen and androgen regiment with intended biological activity for a specific indication selected from frailty, osteoporosis, aging, muscle wasting, hormone replacement, cachexia, atherosclerosis, sexual dysfunction, and cancer prevention.

What is claimed is:

1. A pharmaceutical composition comprising:
a pharmaceutically active amount of a first compound, said first compound being selected from the group of a steroid, a progenitor of said steroid, a regulator of said steroid, a modulator of said steroid receptor, and pharmaceutically acceptable prodrugs or salts thereof,
wherein said first compound has a positive effect on a physiological process related to a steroid biosynthesis, turnover, localization, sensing or action, and
wherein said first compound has a negative effect on at least one of cholesterol homeostasis, lipoprotein homeostasis, and cholesterol-related lipid homeostasis;
a pharmaceutically active amount of a second compound, said second compound being selected from the group of a steroid, a progenitor of said steroid, a regulator of said steroid, a modulator of said steroid receptor, and pharmaceutically acceptable prodrugs or salts thereof, a cholesterol biosynthesis modulator, a cholesterol accumulation modulator, a cholesterol transport modulator, a cholesterol-related lipid biosynthesis modulator, a cholesterol related lipid accumulation modulator, a lipoprotein modulator, and pharmaceutically acceptable prodrugs or salts thereof,
wherein said second compound does not substantially interfere with said positive effect of said first compound; and
wherein said second compound exhibits an effect antagonistic to said negative effect of said first compound; and
at least one pharmaceutically acceptable carrier or diluent.

2. The composition of claim 1, wherein said first compound is selected from the group comprising (a) adrenocorticotropin, (b) aldosterone, (c) an androgenic-anabolic steroid, (d) an androgen, (e) an AR antagonist, (f) a cytochrome b5 (CYB5A) or an activity regulator thereof, (g) DHEA, (h) DHEA sulfate, (i) ethinyl estradiol, (j) estradiol, (k) natural or synthetic estrogen, (l) esterified estrogen, (m) a GnRH modulator, (n) 11-hydroxyandrosterone, 17-hydroxyprogesterone, (o) a 17-ketogenic steroid, (p) levonorgestrel, (q) medroxyprogesterone acetate, (r) a P450-oxidoreductase (POR) or an activity regulator thereof, (s) a P450c17 (CYP17A1) phosphorylation regulator, (t) pregnanediol, (u) pregnenolone, (v) progestin, (w) a steroid hormone receptor modulator, (x) a steroidal androgen, (y) a SERM compound, and (z) an SHBG or SHBG regulator.

3. The composition of claim 1 wherein said first compound exhibits a negative effect on HDL levels, and said second compound increases HDL levels without substantially interfering with the positive effect of said first compound.

4. The composition of claim 3 wherein said first compound is selected from the group of oral androgenic-anabolic steroids, progestins, high-dose isoflavone, cortisol, gonadotropin inhibitors, androgen synthesis inhibitors, aldosterones, SR-BI inhibitors, 21α-hydroxylase inhibitors, 11β-hydroxylase inhibitors, steroid binding globulin inhibitors; and
wherein said second compound is selected from the group including omega-3 acid ethyl ester, statins, oral estrogens, dexamethasone, CETP inhibitors, total testosterone, non-orally administered androgen, corticosteroids, MR agonist inhibitors, GnRH modulators, steroid binding globulins (SHBG and CBG), and endogenous steroid biosynthesis promoters.

5. The composition of claim 1 wherein said first compound is selected form the group of steroids, steroid biosynthesis regulators, steroid stability regulators, steroid localization regulators and steroid signaling-regulating molecules, and said second compound is a selective steroid receptor modulator (SSRM).

6. The composition of claim 5 wherein said first compound is a tissue-specific SARM selected from a list of tissue-specific SARMS that comprises LGD-3303, and said second compound is a SERM compound.

7. The composition of claim 5 wherein said second compound exhibits at least one of the following: heightened liver antagonistic activity, heightened hypothalamic antagonistic activity, heightened pituitary gland antagonistic activity, specific liver antagonistic activity, specific hypothalamic antagonistic activity, and specific pituitary gland antagonistic activity.

8. The composition of claim 5 wherein said first compound is a progesterone and said second compound is an SPRM that reduces a stimulative effect of progesterone on breast tissues without impacting an anti-estrogenic effect of said progesterone in the uterus.

9. The composition of claim 5 wherein said first compound is an estrogen and said second compound is an SSRM that reduces a venous thrombosis negative effect of said first compound.

10. The composition of claim 1, wherein said second compound is a statin.

11. A pharmaceutical composition comprising:
a pharmaceutically active amount of a first compound, said first compound being selected from the group consisting of a cholesterol regulator, a cholesterol-related lipid regulator, a lipoprotein regulator, and pharmaceutically acceptable prodrugs or salts thereof,
wherein said first compound has a positive effect on a physiological process related to at least one of the group of processes including (a) cholesterol biosynthesis, (b) cholesterol turnover, (c) cholesterol localization, (d) cholesterol sensing, (e) cholesterol action, (f) cholesterol-related lipid biosynthesis, (g) cholesterol-related lipid turnover, (h) cholesterol-related lipid localization, (i) cholesterol-related lipid sensing, (j) lipoprotein homeostasis, (k) cholesterol metabolism, and (l) lipoprotein action,
and wherein said first compound has a negative effect on steroid homeostasis;
a pharmaceutically active amount of a second compound, said second compound being selected from the group consisting of (a) a steroid, (b) a progenitor of said steroid, (c) a regulator of said steroid, (d) a regulator of the synthesis or accumulation of said steroid, (e) a regulator of signal transduction related to said steroid, and (f) a modulator of said steroid receptor;
and pharmaceutically acceptable prodrugs or salts thereof;
wherein said second compound does not substantially interfere with said positive effect of said first compound; and
wherein said second compound exhibits an effect antagonistic to said negative effect of said first compound; and
at least one pharmaceutically acceptable carrier or diluent.

12. The composition of claim 11 wherein said first compound is selected from the group comprising (a) a bile acid sequestrant, (b) a cholesterol absorption inhibitor, (c) a cortisol, (d) a CETP inhibitor, (e) dexamethasone, (f) an estrogen or progestrin that impacts HLD levels, (g) a GnRH modulator, (h) an isoflavone, (i) a long-term calorie restriction regime, (j) medroxyprogesterone acetate, (k) an omega-3 acid ethyl ester, and (l) a statin.

13. The composition of claim 12 wherein said first compound is a statin.

14. The composition of claim 11 wherein said second compound is an SSRM.

15. The composition in claim 11 wherein said first compound is a liver-targeting SHBG modulator and said second compound is a compound that enhances SHBG binding to steroids that does not interfere with the positive effect of said first compound, said positive effect being selected from the list of positive effects that comprises increasing HDL levels and increasing HDL efficiency, and said second compound exhibits an effect antagonistic to said negative effect of said first compound of decreasing said endogenous steroid hormone production.

16. The composition of claim 15 wherein the second compound is an SSRM.

17. A method for altering at least one trait among the group including (a) steroid accumulation level, (b) steroid localization, (c) steroid sensing, (d) steroid signal transduction, in at least one cell, tissue, organ or region of a mammal comprising
identifying a mammal having a condition associated with the said at least one trait; and
administering to said mammal a first compound or regimen that alters said accumulation level; and
administering to said mammal a second compound or regimen, wherein said second compound or regimen does not interfere with a desired effect on said first trait, and wherein said second compound exhibits an effect on the biosynthesis, accumulation or transport of cholesterol, HDL or LDL that is antagonistic to the effect of said first compound.

18. The method of claim 17 wherein said first compound is selected from the group comprising (a) adrenocorticotropin, (b) aldosterone, (c) an androgenic-anabolic steroid, (d) an androgen, (e) an AR antagonist, (f) a cytochrome b5 (CYB5A) or an activity regulator thereof, (g) DHEA, (h) DHEA sulfate, (i) ethinyl estradiol, (j) estradiol, (k) natural or synthetic estrogen, (l) esterified estrogen, (m) a GnRH modulator, (n) 11-hydroxyandrosterone, 17-hydroxyprogesterone, (o) a 17-ketogenic steroid, (p) levonorgestrel, (q) medroxyprogesterone acetate, (r) a P450-oxidoreductase (POR) or an activity regulator thereof, (s) a P450c17 (CYP17A1) phosphorylation regulator, (t) pregnanediol, (u) pregnenolone, (v) progestin, (w) a steroid hormone receptor modulator, (x) a steroidal androgen, (y) a SERM compound, and (z) an SHBG or SHBG regulator.

19. The method of claim 17, wherein said second compound is a statin.

20. The method of claim 17 wherein said second compound is an SSRM.

21. The method of claim 20 wherein said second compound has heightened activity in one or more of the regions chosen from the group consisting of the liver, hypothalamus, and pituitary gland of the mammal.

22. The method of claim 17, wherein said steps of administering occur at substantially at the same time.

23. A method for altering at least one trait among the group including (a) cholesterol accumulation level, (b) cholesterol localization, (c) cholesterol sensing, (d) cholesterol signal transduction, and (e) lipoprotein accumulation level, in at least one cell, tissue, organ or region of a mammal comprising
identifying a mammal having a condition associated with said trait; and
administering to said mammal a first compound or regimen that alters said first trait; and
administering to said mammal a second compound or regimen, wherein said second compound or regimen does not interfere with a desired effect on the regulation of said trait, and wherein said second compound exhibits an effect on a second trait selected from the group including (a) steroid biosynthesis, (b) steroid accumulation, (c) steroid transport, (d) steroid signaling and (e) steroid sensing, that is antagonistic to the effect of said first compound.

24. A method for altering at least one trait among the group including (a) cholesterol-related lipid accumulation level, (b) cholesterol-related lipid localization, (c) cholesterol-related lipid sensing, (d) cholesterol-related lipid transport, and (e) lipoprotein accumulation level, in at least one cell, tissue, organ or region of a mammal comprising
identifying a mammal having a condition associated with said first trait; and
administering to said mammal a first compound or regimen that alters said cholesterol-related lipid accumulation level; and
administering to said mammal a second compound or regimen, wherein said second compound or regimen does not interfere with an effect on the regulation of said accumulation level, and wherein said second compound exhibits an effect on steroid biosynthesis, accumulation or transport that is antagonistic to the effect of said first compound.

25. The method of claim 23, wherein said first compound or regimen is selected from the group consisting of (a) a bile acid sequestrant, (b) a cholesterol absorption inhibitor, (c) a cortisol, (d) a CETP inhibitor, (e) dexamethasone, (f) an estrogen or progestrin that impacts lipid levels, (g) a GnRH modulator, (h) an isoflavone, (i) a long-term calorie restriction regime, (j) medroxyprogesterone acetate, (k) an omega-3 acid ethyl ester, and (l) a statin.

26. The method of claim 25, wherein the regulator is a statin.

27. The method of claim 26 wherein said second compound is an SSRM.

28. A method for altering at least one cholesterol accumulation level in at least one cell, tissue, organ or region of a mammal comprising
identifying a mammal having a condition associated with said cholesterol accumulation level; and
administering to said mammal a first compound or regimen that alters said cholesterol accumulation level, wherein said first compound is a steroid synthesis regulator.

29. The method of claim 28 wherein said compound is selected from the group including (a) DHEA, (b) DHEAS, (c) artificial adrenarch, (d) a 17,20-lyase activity regulator, (e) a P450-oxidoreductase (POR), (f) a P450-oxidoreductase (POR) regulator, (g) a cytochrome b5 (CYB5A), (h) a cytochrome b5 (CYB5A) regulator, (i) a P450c17 (CYP17A1) protein kinase, and (j) a P450c17 (CYP17A1) protein kinase regulator.

30. A method of identifying at least one agent of a multi-agent medicament for altering at least one among the group including steroid accumulation level, steroid sensing, steroid localization, steroid action, and steroid signal transduction, in at least one cell, tissue, organ or region of a mammal, said method comprising
administering a first agent to the mammal, wherein the first agent affects at least one member of said group; and
administering a second agent to the mammal,
evaluating whether the second agent counteracts the effect on at least one member of said group, and
evaluating whether the second agent exhibits an effect on a second group comprising cholesterol biosynthesis, cholesterol accumulation, cholesterol transport, cholesterol-related lipid synthesis, cholesterol-related lipid accumulation, lipoprotein homeostasis, and cholesterol-related lipid transport, that is antagonistic to the effect of said first agent.

31. The method of claim 30, wherein the second agent is selected from the group consisting of (a) a non-peptidyl small molecule, (b) a peptide, (c) an antibody, (d) an antisense molecule, (e) a small interfering RNA molecule, (f) a gene, or (g) a stem cell.

32. The method of claim 30, wherein said first compound is selected from the group comprising (a) adrenocorticotropin, (b) aldosterone, (c) an androgenic-anabolic steroid, (d) an androgen, (e) an AR antagonist, (f) a cytochrome b5 (CYB5A) or an activity regulator thereof, (g) DHEA, (h) DHEA sulfate, (i) ethinyl estradiol, (j) estradiol, (k) natural or synthetic estrogen, (l) esterified estrogen, (m) a GnRH modulator, (n) 11-hydroxyandrosterone, 17-hydroxyprogesterone, (o) a 17-ketogenic steroid, (p) levonorgestrel, (q) medroxyprogesterone acetate, (r) a P450-oxidoreductase (POR) or an activity regulator thereof, (s) a P450c17 (CYP17A1) phosphorylation regulator, (t) pregnanediol, (u) pregnenolone, (v) progestin, (w) a steroid hormone receptor modulator, (x) a steroidal androgen, (y) a SERM compound, and (z) an SHBG or SHBG regulator.

33. The method of claim 30 wherein said first molecule affects androgen signaling and said second molecule is an SARM.

34. The method of claim 30 wherein said first molecule affects estrogen signaling and said second molecule is an SERM.

35. The method of claim 30 wherein said first molecule affects progesterone signaling and said second molecule is an SPRM.

36. A compound identified by the process of any of claims 30-34 and 35.

37. A method of identifying at least one agent of a multi-agent medicament for altering at least one cholesterol accumulation level in at least one cell, tissue, organ or region of a mammal, said method comprising
administering a first agent to the mammal, wherein the first agent affects said cholesterol accumulation level; and
administering a second agent to the mammal,
evaluating whether the second agent does not counteract the effect on the regulation of said cholesterol accumulation level of said first compound, and
evaluating whether the second agent exhibits an effect on steroid biosynthesis, accumulation or transport which is antagonistic to the effect of said first agent.

38. A method of identifying at least one agent of a multi-agent medicament for altering at least one cholesterol-related lipid accumulation level in at least on cell, tissue, organ or region of a mammal, said method comprising
administering a first agent to the mammal, wherein the first agent affects at least one member of a first group of traits including cholesterol-related lipid accumulation level, cholesterol-related lipid localization, cholesterol-related lipid sensing, lipoprotein homeostasis, and cholesterol-related lipid signaling; and
administering a second agent to the mammal,
evaluating whether the second agent does not counteract the effect of said first agent on said first group of traits, and
evaluating whether the second agent exhibits an effect on at least one member of a second group of traits including steroid biosynthesis, steroid accumulation, steroid transport, steroid sensing, steroid action, and steroid signaling, wherein said effect of said second agent is antagonistic to the effect of said first agent.

39. The method of any of claims 37 and 38, wherein said regulator is selected from the group consisting of (a) a bile acid sequestrant, (b) a cholesterol absorption inhibitor, (c) a cortisol, (d) a CETP inhibitor, (e) dexamethasone, (f) an estrogen or progestrin that impacts HLD levels, (g) a GnRH modulator, (h) an isoflavone, (i) a long-term calorie restriction regime, (j) medroxyprogesterone acetate, (k) an omega-3 acid ethyl ester, and (l) a statin.

40. A compound identified by the process of any of claims 37-38 and 39.

41. A method of identifying a compound that both (a) modulates a trait selected from the group including cholesterol synthesis, cholesterol turnover, cholesterol transport, cholesterol sensing, cholesterol metabolism, and cholesterol signaling, and (b) modulates a trait selected from the group including steroid biosynthesis, steroid turnover, steroid localization, steroid transport, steroid sensing, and steroid signaling, said method comprising
monitoring the effect of said compound on a trait from said first group;
monitoring the effect of said compound on a trait from said second group; and
identifying the compound.

42. A compound identified by the method of claim 41.

43. A method of identifying an effect of a steroid-modulating compound on cholesterol homeostasis said method comprising
monitoring the effect of said compound on said cholesterol homeostasis.

44. A method of identifying an effect of a steroid turnover regulating compound on cholesterol homeostasis, said method comprising
monitoring the effect of said compound on said cholesterol homeostasis.

45. A method of identifying an effect of a steroid localization regulating compound on cholesterol homeostasis, said method comprising
monitoring the effect of said compound on said cholesterol homeostasis.

46. A method of identifying an effect of a steroid sensing modulating compound on cholesterol homeostasis, said method comprising
monitoring the effect of said compound on said cholesterol homeostasis.

47. The method of claim 43 wherein said steroid-modulating compound is chosen from the group including (a) DHEA, (b) DHEAS, (c) artificial adrenarch, (d) a 17,20-lyase activity regulator, (e) a P450-oxidoreductase (POR), (f) a P450-oxidoreductase (POR) regulator, (g) a cytochrome b5 (CYB5A), (h) a cytochrome b5 (CYB5A) regulator, (i) a P450c17 (CYP17A1) protein kinase, (j) a P450c17 (CYP17A1) protein kinase regulator, and (k) an SSRM.

48. A compound identified by the process of claim 47.

49. A method of identifying an effect of a compound on cholesterol homeostasis, said method comprising
monitoring the effect of said compound on said cholesterol homeostasis,
wherein said compound also modulates steroid synthesis, turnover, transport or sensing.

50. The method of claim 49 wherein said compound is chosen from the group including (a) DHEA, (b) DHEAS, (c) artificial adrenarch, (d) a 17,20-lyase activity regulator, (e) a P450-oxidoreductase (POR), (f) a P450-oxidoreductase (POR) regulator, (g) a cytochrome b5 (CYB5A), (h) a cytochrome b5 (CYB5A) regulator, (i) a P450c17 (CYP17A1) protein kinase, and (j) a P450c17 (CYP17A1) protein kinase regulator, and (k) a SSRM.

51. A compound identified by the process of claim 50.

52. A method of identifying an effect of a compound on SHBG levels, said method comprising monitoring the effect of said compound on SHBG levels, wherein said compound is selected from the group including nuclear receptor ligands, SSRMs, PPAR modulators, and other SHBG regulators.

53. A compound identified by the process of claim 52.

54. A method of identifying an effect of a molecule on endogenous sex steroid production comprising screening said molecule in an SHBG binding assay for an enhanced binding of an SHBG to a hormone, wherein said increased binding leads to a higher availability of cholesterol in steroidogenic tissues for steroid biosynthesis.

55. A compound identified by the process of claim 54.

56. A method of identifying an effect of a steroid-modulating compound on lipoprotein homeostasis said method comprising
monitoring the effect of said compound on said lipoprotein homeostasis.

57. A method of identifying an effect of a steroid turnover regulating compound on lipoprotein homeostasis said method comprising
monitoring the effect of said compound on said lipoprotein homeostasis.

58. A method of identifying an effect of a steroid localization regulating compound on lipoprotein homeostasis said method comprising
monitoring the effect of said compound on said lipoprotein homeostasis.

59. A method of identifying an effect of a steroid sensing modulating compound on lipoprotein homeostasis said method comprising
monitoring the effect of said compound on said lipoprotein homeostasis.

60. The method of claim 56 wherein said compound is chosen from the group including (a) DHEA, (b) DHEAS, (c) artificial adrenarch, (d) a 17,20-lyase activity regulator, (e) a P450-oxidoreductase (POR), (f) a P450-oxidoreductase (POR) regulator, (g) a cytochrome b5 (CYB5A), (h) a cytochrome b5 (CYB5A) regulator, (i) a P450c17 (CYP17A1) protein kinase, (j) a P450c17 (CYP17A1) protein kinase regulator, and (k) an SSRM.

61. A compound identified by the process of claim 60.

62. A method of identifying an effect of a compound on lipoprotein homeostasis, said method comprising
monitoring the effect of said compound on said lipoprotein homeostasis,
wherein said compound also modulates steroid synthesis, turnover, transport or sensing.

63. The method of claim 62 wherein said compound is chosen from the group including (a) DHEA, (b) DHEAS, (c) artificial adrenarch, (d) a 17,20-lyase activity regulator, (e) a P450-oxidoreductase (POR), (f) a P450-oxidoreductase (POR) regulator, (g) a cytochrome b5 (CYB5A), (h) a cytochrome b5 (CYB5A) regulator, (i) a P450c17 (CYP17A1) protein kinase, and (j) a P450c17 (CYP17A1) protein kinase regulator, and (k) a SSRM.

64. A compound identified by the process of claim 63.
